(19) United States
(12) Patent Application Publication
(10) Pub. No.: US 2003/0171315 A1
(43) Pub. Date: Sep. 11, 2003

(54) Oligonucleotide probes and primers comprising universal bases for therapeutic purposes

(76) Inventors: Bob D. Brown, Encinitas, CA (US); Timothy A. Riley, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/142,666

(22) Filed: May 8, 2002

Related U.S. Application Data
(60) Provisional application No. 60/306,229, filed on Jul. 18, 2001.

(51) Int. Cl. A61K 48/00; C07H 21/04; C12N 15/85

(52) U.S. Cl. 514/44; 435/455; 536/23.2

(57) ABSTRACT

Aspects of the invention relate novel oligonucleotides comprising universal and/or generic bases, in particular juxtaposed universal and/or generic bases, which can be used to treat or prevent disease.

### RELATED APPLICATIONS

This application claims priority to U.S. Provisional Application No. 60/306229, filed Jul. 18, 2001. This application also claims priority to Application Ser. No. 09/136,080 filed on Aug. 18, 1998, which claimed priority from U.S. Provisional Application No. 60/060,673 filed on Oct. 2, 1997.

### FIELD OF THE INVENTION

Aspects of the invention relate novel oligonucleotides comprising universal and/or generic bases, in particular juxtaposed universal and/or generic bases, which can be used to treat or prevent disease.

### BACKGROUND OF THE INVENTION

The explosion of recent knowledge in basic genetics has spawned numerous clinical follow-up studies that have confirmed an unequivocal association between the presence of specific prevalent genetic alterations and susceptibility to some very common human diseases. In addition, the Human Genome Project's sequencing efforts will contribute yet more candidate disease genes that will require both research-based genetic association studies (to confirm suspected disease links) and, if positive, the translation of these disease-genotype associations to routine diagnostic clinical practice. The knowledge of which genes are associated with disease also allows for the development of molecular approaches to treating and preventing disease.

Antisense oligonucleotides have received considerable attention for their potential use as the “silver bullet” of pharmacological agents and, in the last few years, therapeutics containing antisense oligonucleotides have begun to enter the market. In 1998 the Food and Drug Administration approved the first drug containing an antisense oligonucleotide directed to cytomegalovirus (CMV) retinis, a virus that infects the human eye in many AIDS patients and others whose immune system is depressed resulting in blindness. Marketed as Vitravene, the therapeutic is administered by direct injection into the eye whereby the active ingredient interferes with the replication mechanism of the retina-destroying cytomegalovirus.

Central to the effectiveness of an antisense oligonucleotide therapeutic the ability of the active ingredient to hybridize to its target with a high degree of specificity. Accordingly, many in the field have endeavored to identify methods to increase the specificity and affinity of oligonucleotides for their targets. Various methods for increasing the specificity of oligonucleotides are known in the art, including increasing the length, choosing oligonucleotides that are not likely to cross-hybridize or bind non-specifically and designing oligonucleotides that have a high annealing temperature. (See e.g., Bergstrom et al., J. Am. Chem. Soc. 117:1201-1209, 1995; Nicols et al., Nature 369:4920493, 1994; Loakes, Nucl. Acids Res. 22:4039-4043, 1994; Brown, Nucl. Acids Res. 20:5149-5152, 1992).

Recently, investigators have determined that modified oligonucleotides containing universal bases provide some benefit over conventional oligonucleotide chemistries. (See Guo et al., U.S. Pat. No. 5,870,233, filed Jun. 6, 1996). Although Guo et al., observed some improvement in being able to discriminate a variant nucleotide in a target nucleic acid by incorporating solitary universal bases (artificial mismatches) sprinkled throughout a probe oligonucleotide, particular spacing and composition requirements were necessary. For example, Guo et al. found that the universal base should be carefully spaced from the variant nucleotide (i.e., 3 or 4 nucleotides away) and that the oligonucleotide probes should not contain a total composition of universal bases of greater than 15%.

Van Ness et al. (U.S. Pat. No. 6,361,940, filed Apr. 1, 1998) also found that the incorporation of universal bases (specificity spacers) could increase the specificity of a probe oligonucleotide for a target nucleic acid. As above, however, Van Ness et al. determined that the universal bases should be spaced a considerable distance from each other (4-14 nucleotides). Despite the many advances made in the field, there still remains a need for better oligonucleotide chemistries, which allow for the development of more efficient therapeutics.

### SUMMARY OF THE INVENTION

Aspects of the invention concern antisense oligonucleotides having universal and/or generic bases, preferably in a juxtaposed position, which can be used to treat and prevent disease. It was discovered that oligonucleotides having a universal and/or generic base composition of at least 20%-30% of the total number of bases exhibit a high degree of specificity for their target. Further, it was discovered that oligonucleotides having at least 2, 3, 4, 5, 6, 7, 8, 9, 10, 11, 12, 13 or more juxtaposed (side-by-side) universal and/or generic bases exhibited a high degree of specificity for their target. The oligonucleotides described herein are well suited for therapeutic uses, such as antisense approaches to prevent or treat cancer, inflammation, tumor development, and cell senescence because the universal or generic bases increase the specificity for a target and concomitantly increase the recruitment of RNases to the target (e.g., RNase H).

Embodiments include, for example, an improved antisense oligonucleotide, which comprises a domain that recruits an RNase and inhibits the function of a gene associated with a human disease, wherein the improvement comprises the incorporation of at least 2, 3, 4, 5, or 6 juxtaposed universal bases in said oligonucleotide.

Embodiments also include a method of inhibiting the function of a gene associated with a human disease comprising contacting a cell containing said gene with the improved antisense oligonucleotide above, whereby the function of the gene in said cell is inhibited.

Embodiments also include a method of inhibiting the function of a gene associated with a human disease comprising providing an antisense oligonucleotide, which comprises a domain that recruits an RNase and at least 2, 3, 4, 5, 6, 7, or 8 juxtaposed universal bases and contacting a cell that expresses said gene with said antisense oligonucleotide, whereby said contact inhibits the function of said gene.

### BRIEF DESCRIPTION OF THE DRAWINGS

FIG. 1 shows the detection of a single nucleotide base change by quantification of melting temperatures.

### DETAILED DESCRIPTION OF THE PREFERRED EMBODIMENT

Aspects of the invention concern antisense oligonucleotides that contain universal and/or generic bases or other unnatural bases, preferably in a juxtaposed position so as to improve the specificity of the oligonucleotide for its target, and methods of using these improved oligonucleotides to treat or prevent disease. The improvements described herein are generally applicable to antisense technology and are readily adaptable for use with antisense strategies in all organisms in which conventional antisense techniques can be applied including, but not limited to, plants, animals, mammals, insects, fungi, mold, and nematodes.

It was discovered that the specificity of an oligonucleotide and the ability to perform antisense inhibition of a gene was improved by incorporating 2 or more juxtaposed nucleic acids with universal bases. In a first set of experiments, it was observed that the incorporation of a block of juxtaposed universal bases in an oligonucleotide facilitated the differentiation of nucleic acids that differed by as little as a single nucleotide. Accordingly, by incorporating blocks of universal bases into the molecules, highly specific oligonucleotides were developed. In fact, it was found that the presence of five universal bases within an oligonucleotide having a single base mismatch with a target molecule decreased the melting temperature of probe-template hybrids by 17° C., in comparason to an oligonucleotide with no mismatches. Moreover, conventional oligonucleotides that had a single mismatch with a target molecule only had a 6° C. decrease in melting temperature.

In a second set of experiments it was discovered that the incorporation of a block of universal bases (two juxtaposed universal bases) in an oligonucleotide exhibited significant antisense inhibition of a B cell lymphoma-associated gene (BCL2) in the T-24 cell line. In other experiments, it was found that several different antisense oligonucleotides containing large blocks of universal and/or generic bases (e.g., blocks of more than 5 juxtaposed artificial bases) were effectively taken up by A549 cells (a human melanoma cell line) and significant antisense activity was detected.

Embodiments of the invention include oligonucleotides that contain greater than a 20% composition of universal and/or generic bases, wherein the bases contained in the oligonucleotide are stacked side-by-side into blocks (“juxtaposed”) of 2, 3, 4, 5, 6, 7, 8, 9, 10, 11, 12, 13 or more universal and/or generic bases. Embodiments also include oligonucleotides having at least 21%, 22%, 23%, 24%, 25%, or 30% universal, generic or a mixture of universal and generic bases, preferably in blocks of juxtaposed artificial bases. Still more embodiments are oligonucleotides with at least 31%, 32%, 33%, 34%, 35%, 36%, 37%, 38%, 39%, 40%, 45%, 50%, 55%, 60%, or more universal, generic or a mixture of universal and generic bases and unnatural bases, wherein said universal and/or generic bases are, preferably, in one or more blocks of juxtaposed artificial bases.

In some contexts, the term “universal base” is used to describe a moiety that may be substituted for any nucleic acid base. The universal base need not contribute to hybridization, but should not significantly detract from hybridization, whereas “generic bases” are bases that are capable of binding to more than one type of nucleotide. For example a base might be generic for the purine bases or alternatively a base might be generic for the pyrimidine bases. Preferred universal or generic bases include 2-deoxyinosine, 5-nitroindole, 3-nitropyrrole, 2-deoxynebularine, dP, or dK derivatives of natural nucleotides. Some embodiments may also utilize degenerate bases. The term “degenerate base” refers to a moiety that is capable of base-pairing with either any purine, or any pyrimidine, but not both purines and pyrimidines. Exemplary degenerate bases include, but are not limited to, 6H, 8H-3,4-dihydropyrimido[4,5-c][1,2]oxazin-7-one (“P”, a pyrimidine mimic) and 2-amino-6-methoxyaminopurine (“K”, a purine mimic). In some aspects of the invention, these universal, generic, or degenerate bases are juxtaposed in blocks of artificial bases and in others, they are clustered at either the 5′ or 3′ end of the oligonucleotide or both. Desirably, at least 2, 3, 4, 5, 6, 7, 8, 9, or 10, 11, 12, 13, or more universal, generic, or degenerate bases are juxtaposed in each block and an oligonucleotide may contain 1, 2, 3, 4, 5, 6, 7, 8, 9, or 10 blocks depending on the length of the oligonucleotide and the desired effect. Further, some embodiments contain a non-nucleic acid linker such as a spacer 9, spacer 18, spacer C3, or a dSpacer so as to provide greater flexibility in the molecule. In some contexts, these spacers are also referred to as universal bases.

The oligonucleotides described herein may also contain natural bases or unnatural base analogs that hydrogen bond to natural bases in the target nucleic acid. Additionally, the oligonucleotides described herein may contain natural bases or unnatural base analogs or other modifications that have a lower affinity to or ability to hydrogen bond to natural bases, relative to any natural base. By “non-naturally occurring base” is meant a base other than A, C, G, T and U, and includes degenerate and universal bases as well as moieties capable of binding specifically to a natural base or to a non-naturally occurring base. Non-naturally occurring bases include, but are not limited to, propynylcytosine, propynyluridine, diaminopurine, 5-methylcytosine, 7-deazaadenosine and 7-deazaguanine. In still more embodiments, the oligonucleotides described above have at least two high affinity domains and one or more low affinity domains.

Embodiments of the invention also include methods of making and using the oligonucleotides described above. For example, one embodiment concerns a method of designing an oligonucleotide, which involves identifying a sequence that corresponds to, or complements, a target sequence and substituting two or more bases, preferably two or more juxtaposed bases, within said sequence with universal or generic bases. Another embodiment concerns a method of increasing the specificity of an oligonucleotide by substituting at least 20%, 21%, 22%, 23%, 24%, 25%, 26%, 27%, 28%, 29%, 30%, 31%, 32%, 33%, 34%, 35% or 40% of the total number of bases with universal or generic bases. Preferably, said substitutions are made such that blocks of juxtaposed (side-by-side stacks) universal and/or generic bases are created A further embodiment concerns a method of increasing the specificity of an oligonucleotide by substituting at least 35%, 40%, 45%, 50%, 55%, 60%, or 70% of the total number of bases with universal or generic bases.

Aspects of the invention also include approaches to treat and/or prevent disease. Particularly desirable embodiments concern the treatment and prevention of various types of cancer, inflammation, diseases associated with abnormal cell senescence, and TNF-α or STAT-3 associated diseases. In one embodiment, for example, an approach for the treatment and/or prevention of B cell lymphoma is provided. A subject in need of a medicament for the treatment and/or prevention of B cell lymphoma is identified and said subject is provided a therapeutically or prophylactically effective amount of a pharmaceutical comprising an antisense oligonulceotide that complements the BCL-2 gene, wherein said oligonucleotide comprises at least two juxtaposed (side-by-side block) universal and/or generic bases. Optionally, the subject is monitored for the effectiveness of antisense inhibition of the BCL-2 gene. The antisense oligonucleotides can have a sequence that corresponds to the 5′ untranslated region, 3′ untranslated region, coding region, start region, or stop region. Additionally, combinations of antisense oligonucleotides that correspond to various different regions of the gene can be used.

In another embodiment, an approach to treat or prevent melanoma is provided. Accordingly, a subject in need of a medicament to treat and/or prevent melanoma is identified and said subject is provided a therapeutically or prophylactically effective amount of a pharmaceutical comprising an antisense oligonucleotide that complements one or more of the following genes: STLK4, PTP-α, ZC1, GSK3β, and HRI, wherein said oligonucleotide comprises at least two juxtaposed (side-by-side block) universal and/or generic bases. Optionally, the subject is monitored for the effectiveness of antisense inhibition of one or more of the genes above. The antisense oligonucleotides can have a sequence that corresponds to the 5′ untranslated region, 3′ untranslated region, coding region, start region, or stop region or any intron sequence of any one of the genes above. Additionally, combinations of antisense oligonucleotides that correspond to various different regions of one or more of the genes above can be used.

In another embodiment, an approach to treat or prevent myeloma, breast carcinoma, brain tumors, leukemia, and other cancers associated with over expression of STAT-3 is provided. Accordingly, a subject in need of a medicament for the treatment and/or prevention of a cancer associated with the over expression of STAT-3 is identified and said subject is provided a therapeutically or prophylactically effective amount of a pharmaceutical comprising an antisense oligonulceotide that complements the STAT-3 gene, wherein said oligonucleotide comprises at least two juxtaposed (side-by-side block) universal and/or generic bases. Optionally, the subject is monitored for the effectiveness of antisense inhibition of the STAT-3 gene. The antisense oligonucleotides can have a sequence that corresponds to the 5′ untranslated region, 3′ untranslated region, coding region, start region, or stop region. Preferably, the antisense oligonucleotides complement sequences in the 3′ untranslated region. Additionally, combinations of antisense oligonucleotides that correspond to various different regions of the gene can be used.

In still another embodiment, an approach to treat or prevent breast cancer is provided in which a subject in need of a medicament for the treatment and/or prevention of breast cancer is identified and said subject is provided a therapeutically or prophylactically effective amount of a pharmaceutical comprising an antisense oligonulceotide that complements the HER-2 gene, wherein said oligonucleotide comprises at least two juxtaposed (side-by-side block) universal and/or generic bases. Optionally, the subject is monitored for the effectiveness of antisense inhibition of the HER-2 gene. The antisense oligonucleotides can have a sequence that corresponds to the 5′ untranslated region, 3′ untranslated region, coding region, start region, or stop region of the HER-2 gene. Preferably, the antisense oligonucleotides complement sequences in the coding region. Additionally, combinations of antisense oligonucleotides that correspond to various different regions of the gene can be used.

In another embodiment, a method of inhibiting the progression of cancer is provided in which a subject in need of a medicament for the treatment and/or prevention of cancer is identified and said subject is provided a therapeutically or prophylactically effective amount of a pharmaceutical comprising an antisense oligonulceotide that complements the focal adhesion kinase (FAK, also pp125FAK) gene, wherein said oligonucleotide comprises at least two juxtaposed (side-by-side block) universal and/or generic bases. Optionally, the subject is monitored for the effectiveness of antisense inhibition of the FAK gene. The antisense oligonucleotides can have a sequence that corresponds to the 5′ untranslated region, 3′ untranslated region, coding region, start region, or stop region of the FAK gene. Preferably, the antisense oligonucleotides complement sequences in the coding region. Additionally, combinations of antisense oligonucleotides that correspond to various different regions of the gene can be used.

In another embodiment, a therapeutically effective amount of an antisense oilgonucleotide, which complements a region of TNF-α is provided to subject in need of a medicament to treat inflammation. The administered oligonucleotide comprises at least two juxtaposed (side-by-side) universal and/or generic bases, referred to as a block of artificial bases, which improves the specificity of the oligonucleotide for its target and increases the ability to conduct antisense inhibition (e.g., by recruiting RNase H). The antisense sequence is designed from the cDNA sequence published by Nedwin, G. E. et al. (Nucleic Acids Res. 1985, 13, 6361-6373), herein expressly incorporated by reference. Although sequences within the 5′ untranslated region, 3′ untranslated region, coding region, start region, or stop region can be used as targets, particularly desirable sequences correspond to the stop codon and the start site. (See Hartmann, G., et al., Antisense Nucleic Acid Drug Dev., 1996, 6, 291-299, which describes a TNF-α antisense oligodeoxynucleotide targeted to the start site of the TNF-α gene), herein expressly incorporated by reference.

In another embodiment, an approach to treat and/or prevent cell senescence is provided in which a subject in need of a medicament for the treatment and/or prevention of a disease associated with abnormal cell senescence is identified and said subject is provided a therapeutically or prophylactically effective amount of a pharmaceutical comprising an antisense oligonulceotide that complements a senescent cell derived inhibitor (SDI) gene, wherein said oligonucleotide comprises at least two juxtaposed (side-by-side block) universal and/or generic bases. Optionally, the subject is monitored for the effectiveness of antisense inhibition of the SDI gene. The antisense oligonucleotides can have a sequence that corresponds to the 5′ untranslated region, 3′ untranslated region, coding region, start region, or stop region of the SDI gene. Preferably, the antisense oligonucleotides complement sequences in the coding region. Additionally, combinations of antisense oligonucleotides that correspond to various different regions of the gene can be used. The section below describes the oligonucleotides of the invention in greater detail.

Oligonucleotides

The oligonucleotides of the invention can be of virtually any sequence and of any length, wherein said oligonucleotides comprise at least 20%, 21%, 22%, 23%, 24%, 25%, 26%, 27%, 28%, 29%, or 30% or more universal and/or generic bases. The term “oligonucleotide” is used to refer to a molecule consisting of DNA, RNA, or DNA/RNA hybrids with or without non-nucleic acid analogues and polymers. In some embodiments the universal or generic bases are juxtaposed (side-by-side in blocks) and, in others, clusters of at least two universal or generic bases are sprinkled throughout the oligonucleotide sequence. Preferred sequences correspond to already existing antisense oligonucleotides, which have been identified as having therapeutic or prophylactic application. Preferred sequences, for example, include sequences identified as having efficacy in the inhibition of STAT-3 (See e.g., U.S. Pat. No. 6,159,694; hereby expressly incorporated by reference in its entirety), TNF-α (See e.g., U.S. Pat. No. 6,228,642; hereby expressly incorporated by reference in its entirety), HER-2 (See e.g., U.S. Pat. No. 5,968,748; hereby expressly incorporated by reference in its entirety), FAK (See e.g., U.S. Pat. No. 6,133,031; hereby expressly incorporated by reference in its entirety); and SDI (See e.g., U.S. Pat. No. 5,840,845; hereby expressly incorporated by reference in its entirety). It should be understood that other sequences known by those of skill in the art, which indicate a predilection to disease can be used to generate the oligonucleotides of the invention.

By “antisense oligonucleotide” is meant a nucleic acid or modified nucleic acid including, but not limited to DNA, RNA, modified DNA or RNA (including branched chain nucleic acids and 2′ O-methyl RNA) and PNA (polyamide nucleic acid). The antisense oligonucleotides described herein can be of single unit (e.g., a single linear antisense oligonucleotide) or a multi-unit construction, wherein, for example, an “anchor,” a first oligonucleotide comprising a region that complements a target) resides on a separate oligonucleotide from an effector (e.g., a “cleaver”, which causes the target to be cleaved) and the two or more oligonucleotides are joined by a covalent or non-covalent coupling moeity. The term “coupling moiety” as used herein refers to a reactive chemical group that is capable of reacting with another coupling moiety to join two molecules. The coupling moieties used in the invention preferably bind in the absence of any target molecule, and are preferably selected such that the first coupling moiety reacts only with the second coupling moiety, and not with any other portion of the molecule or other first coupling moieties. Similarly, the second coupling moiety should react only with the first coupling moieties, and not with any other second coupling moiety (or any other portion of the molecules).

Exemplary coupling moieties include complementary oligonucleotides (preferably selected such that they do not hybridize to any portion of the target polynucleotide), complementary oligonucleotide analogs (particularly employing bases which do not hybridize to natural bases), and electrophilic or nucleophilic moieties such as alkyl halides, alkyl sulfonates, activated esters, ketones, aldehydes, amines, hydrazines, sulfhydryls, alcohols, phosphates, thiophosphates, Michael addition receptors, dienophiles, dienes, dipolarophiles, nitriles, thiosemicarbazides, imidates, isocyanates, isothicyanates, alkynes, and alkenes. Where the antisense constructs comprise more than two component parts (for example, where three or four molecules are coupled to make the final construct), the coupling moieties are preferably selected such that the first and second coupling moieties react only with each other, and the third and fourth coupling moieties react only with each other, and so forth.

In one embodiment, for example, the coupling moieties are complementary oligonucleotides. The complementary regions can be separated by several non-complementary bases, to provide an inherent flexible linker. The term “stem” as used herein refers to the structure formed by coupling two oligonucleotide or oligonucleotide analog coupling moieties. The complementary oligonucleotides can be attached to the binding domains in the same polarity or orientation, or can be provided in reverse polarity or orientation. For example, where the binding domain is in the 5′-3′ orientation, the complementary oligonucleotide coupling moiety can be attached in the 3′-5′ orientation, thus reducing the chances that the coupling moiety will inadvertently participate (or interfere with) binding to the target polynucleotide. In another embodiment, the oligonucleotide comprises unnatural bases which do not hybridize with natural bases.

The coupling moieties may also join as the result of covalent chemical interactions, for example, by condensation, cycloaddition, or nucleophilic-electrophilic addition. In one embodiment, one coupling moiety can be a sulfhydryl group, while its complementary coupling moiety is a succinimidyl group. In another embodiment, one coupling moiety is an amine or a hydrazine moiety, while the complementary coupling moiety is a carbonyl group (aldehyde, ketone, or activated ester). In another embodiment, one coupling moiety is a maleimidyl group while the complementary coupling moiety is a sulfhydryl group. In another embodiment, one coupling moiety is an aryl-dihydroxyboron group which binds to adjacent OH groups on ribose. In another embodiment, an oxazole derivative forms one coupling moiety, while its complement comprises a diketotriazole, as described by T. Ibata et al., Bull Chem Soc Japan (1992) 65:2998-3007, herein expressly incorporated by reference in its entirety.

Flexible linkers are optionally used to relieve stress that might otherwise result from interposing the coupling moieties between two binding domains that bind to adjacent regions of target nucleic acid. The term “flexible linker” refers to a moiety capable of covalently attaching a binding domain to a coupling moiety. Suitable flexible linkers are typically linear molecules in a chain of at least one or two atoms, more typically an organic polymer chain of 1 to 12 carbon atoms (and/or other backbone atoms) in length. Exemplary flexible linkers include polyethylene glycol, polypropylene glycol, polyethylene, polypropylene, polyamides, polyesters, and the like. The flexible linker is preferably selected to be flexible, hydrophilic, and of sufficient length that the bulk of the coupling moieties does not interfere with hybridization, RNase recognition, and/or RNase activity on the complex. It is preferred, but not essential, to employ a flexible linker between each binding domain and its coupling moiety. It is preferred to employ a linker at least between the binding domain and coupling moiety that serves as an RNase substrate, and more preferred to employ flexible linkers in each oligomer. The linker may be connected to the terminal base of the binding domain, or can be connected one or more bases from the end. Suitable flexible linkers are typically linear molecules in a chain of at least one or two atoms, more typically an organic polymer chain of 1 to 12 carbon atoms (and/or other backbone atoms) in length. Flexible linkers also include additional bases, not complementary to the target sequence. Exemplary flexible linkers include polyethylene glycol, polypropylene glycol, polyethylene, polypropylene, polyamides, polyesters, and the like.

In some embodiments, the antisense oligonucleotides also comprise a region that recruits an RNase, preferably a RNaseH or RNase L recruiting domain. Many such domains are known in the art but, in general, where RNase activity is desired, a backbone capable of serving as an RNase substrate is employed for at least a portion of the oligomer. For example, oligonucleotides having only standard (“natural”) bases and backbones in general contain at least 9, 10, 11, 12, 13, 14, 15, 16, 17, 18, 19, or 20 or more bases in order to bind with sufficient energy to effectively down-regulate gene expression by activating RNase H. Oligonucleotides useful for recruiting RNase L can be prepared by substituting 2′-OMe phosphoramidites for the deoxy amidites used after a spacer (e.g., spacer 9). The resulting oligonucleotide has a 2′-OMe diester portion at the 3′ side of the spacer, and a 2′-OMe phosphorothioate on the 5′ side of the spacer. A linker attached to oligo 2′-5′ adenosine can be attached to the 5′ end of the oligo, as described by Torrence et al., U.S. Pat. No. 5,583,032, and U.S. Pat. No. 5,677,289, both incorporated herein by reference. The product can be purified as described by Torrence et al.

The antisense nucleic acids should have a length and melting temperature sufficient to permit formation of an intracellular duplex having sufficient stability to inhibit the expression of the mRNA in the duplex. Strategies for designing antisense nucleic acids suitable for use in gene therapy are disclosed in Green et al., Ann. Rev. Biochem., 55:569-597 (1986) and Izant and Weintraub, Cell, 36:1007-1015 (1984). In some strategies, antisense molecules are obtained from a nucleotide sequence encoding PVCG1O by reversing the orientation of the coding region with respect to a promoter so as to transcribe the opposite strand from that which is normally transcribed in the cell.

Antisense molecules may be produced by selecting at least one target molecule selected from the group consisting of genes, genomic flanking regions, mRNAs and proteins known to be associated with at least one disease or condition; obtaining RNAs selected from the group consisting of RNAs corresponding to the genes, to genomic flanking regions, initiation codon, intron-exon borders and the like, or the entire sequence of RNAs, including non-coding RNA segments, the 5′-end and the 3′-end, e.g., the poly-A segment and oligos targeted to the juxta-section between coding and non-coding regions, and RNA segments encoding the target proteins; selecting a segment of a first RNA which is at least about 60% homologous to a segment of at least a segment of a second RNA; and synthesizing one or more anti-sense oligonucleotide(s) to the one or more RNA segments.

Although the specific length of the oligonucleotide is determined by the target's length, the anti-sense oligonucleotide(s) are preferably greater than about 7 nucleotides long, and up to about 60 nucleotides long, and longer. The specific backbone chemistry may be selected by an artisan based on the teachings provided here and the knowledge of the art at large. “Non-natural” oligonucleotide analogs, for example, include at least one base or backbone structure that is not found in natural DNA or RNA. Exemplary oligonucleotide analogs include, without limitation, DNA, RNA, phosphorothioate oligonucleotides, peptide nucleic acids (“PNA”s), methoxyethyl phosphorothioates, oligonucleotides containing deoxyinosine or deoxy 5-nitroindole, and the like. The term “backbone” refers to a generally linear molecule capable of supporting a plurality of bases attached at defined intervals. Preferably, the backbone will support the bases in a geometry conducive to hybridization between the supported bases and the bases of a target polynucleotide. One factor that impinges on the selection of the nucleotide bridging residues is the level of nuclease resistance desired and other factors specific to one or the other method of administration. Another factor is the need for localization of the treatment, to minimize or fully avoid side effects which might otherwise be caused along with the therapeutic effect of the antisense molecules.

Oligonucleotide synthesis is well known in the art, as is synthesis of oligonucleotides containing modified bases and backbone linkages. In fact, such oligonucleotides can often be obtained from commercial suppliers upon providing the supplier with the specific sequence and composition information and a request for custom production. Although the preferred length of the oligonucleotides is less than 100 bases, embodiments can be from about 5 to about 500 nucleotides in length, desirably, 10 to about 300 nucleotides in length, more desirably 12 to about 200 nucleotides in length, preferably, 15 to about 100 nucleotides, more preferably 17 to about 50 nucleotides, and most preferably, about 20 to about 40 nucleotides in length.

The oligonucleotides can employ any backbone and any sequence capable of resulting in a molecule that hybridizes to target DNA and/or RNA. Examples of suitable backbones include, but are not limited to, phosphodiesters and deoxyphodiesters, phosphorothioates and deoxypbosphorothioates, 2′-O-substituted phosphodiesters and deoxy analogs, 2′-O-substituted phosphorothioates and deoxy analogs, morpholino, PNA (U.S. Pat. No. 5,539,082, hereby expressly incorporated by reference in its entirety), deoxymethyphosphonates, 2′-O-alkyl methylphosphonates, 3′-amidates, MMI, alkyl ethers (U.S. Pat. No. 5,223,618, hereby expressly incorporated by reference in its entirety) and others as described in U.S. Pat. Nos. 5,378,825, 5,489,677 and 5,541,307, all of which are hereby expressly incorporated by reference in its entirety. Where RNase activity is desired, a backbone capable of serving as an RNase substrate is employed for at least a portion of the oligonucleotide.

Universal or generic bases suitable for use with the embodiments described herein include, but are not limited to, deoxy 5-nitroindole, deoxy 3-nitropyrrole, deoxy 4-nitrobenzimidazole, deoxy nebularine, deoxyinosine, 2′-Ome inosine, 2′-Ome 5-nitorindole, 2′-Ome 3-nitropyrrole, 2′-F inosine, 2′-F nebularine, 2′-F 5-nitroindole, 2′-F 4-nitrobenzimidazole, 2′-F 3-nitropyrrole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′O-methoxyethyl nebularine, 2′-O-methoxyethyl 5-nitroindole, 2′-O-methoxyethyl 4-nitro-benzimidazole, 2′-O-methoxyethyl 3-nitropyrrole, deoxy RₚMP-5-nitroindole dimer 2′-Ome RₚMP-5-nitroindole dimer and the like.

Many of the embodied oligonucleotides are characterized in that they share the formula: “XRY”, wherein “X” consists of about 2-3, 2-5, 5-10, 11-20, or 5-20 modified nucleic acid bases; “R” consists of about 2, 3, 4, 5, 6, 7, 8, 9, 10, or 2-20 juxtaposed universal or generic bases; and “Y” consists of about 3-5, 6-10, 11-15, or 3-20 nucleic acid bases; wherein X, R, and Y are joined and at least 20%, 21%, 22%, 23%, 24%, 25%, 26%, 27%, 28%, 29%, or 30% of the total number of bases are universal or generic bases and X and/or Y might contain a natural or unnatural base and X and/or Y might contain higher or lower affinity bases or analogues.

Other embodiments include oligonucleotides with the formula: “XYY”, wherein “X” consists of about 2-3, 2-5, 5-10, 11-20, 21-30, 31-40, 41-50, or 5-50 modified nucleic acid bases or base analogs that have a lower affinity than natural bases; “R” consists of about 2, 3, 4, 5, 6, 7, 8, 9, 10, or 2-20 juxtaposed universal or generic bases; and “Y′ consists of about 5-10, 11-20, 21-30, 31-40, 41-50, or 5-50 nucleic acid bases; wherein X, R, and Y are joined and at least 20%, 21%, 22%, 23%, 24%, 25%, 26%, 27%, 28%, 29%, or 30% of the total number of bases are universal or generic bases.

Still other embodied oligonucleotides have the formula: “XRZRY”, wherein “X” consists of about 2-3, 2-5, 5-10, 11-20, 21-30, 31-40, 41-50, or 5-50 nucleic acid bases; “R” consists of about 3-5, 6-10, 11-15, 16-20, or 3-20 juxtaposed universal or generic bases; “Z” consists of about 2, 3, 4, 5, 6, 7, 8, 9, 10, or 2-20 modified nucleic acid bases; and “Y” consists of about 5-10, 11-20, 21-30, 31-40, 41-50, or 5-50 nucleic acid bases; wherein X, R, Z, and Y are joined and at least 20%, 21%, 22%, 23%, 24%, 25%, 26%, 27%, 28%, 29%, or 30% of the total number of bases are universal or generic bases.

Still other embodied oligonucleotides have the formula: “XZRZY”, wherein “X” consists of about 2-3, 2-5, 5-10, 11-20, 21-30, 31-40, 41-50, or 5-50 nucleic acid bases; “R” consists of about 2, 3, 4, 5, 6, 7, 8, 9, 10, or 2-20 juxtaposed universal or generic bases; “Z” consists of about 5-10, 11-20, or 5-20 modified nucleic acid bases, which have a lower or higher affinity than natural bases; and “Y” consists of about 5-10, 11-20, 21-30, 31-40, 41-50, or 5-50 nucleic acid bases; wherein X, R, Z, and Y are joined and at least 20%, 21%, 22%, 23%, 24%, 25%, 26%, 27%, 28%, 29%, or 30% of the total number of bases are universal or generic bases.

More embodied oligonucleotides have the formula: “XZXRXZX”, wherein “X” consists of about 2-3, 2-5, 5-10, 11-20, 21-30, 31-40, 41-50, or 5-50 nucleic acid bases; “R” consists of about 2, 3, 4, 5, 6, 7, 8, 9, 10, or 2-20 juxtaposed universal or generic bases; “Z” consists of about 5-10, 11-20, or 5-20 modified nucleic acid bases, which have a lower or higher affinity compared to natural bases; wherein X, R, and Z are covalently joined and at least 20%, 21%, 22%, 23%, 24%, 25%, 26%, 27%, 28%, 29%, or 30% of the total number of bases are universal or generic bases.

Still more embodied oligonucleotides have the formula: “XZXRY”, wherein “X” consists of about 5-10, 11-20, 21-30, 31-40, 41-50, or 5-50 nucleic acid bases; “R” consists of about 3-5, 6-10, 11-15, 16-20, or 3-20 juxtaposed universal or generic bases; “Z” consists of about 5-10, 11-20, or 5-20 modified nucleic acid bases, which have a lower or higher affinity than natural bases; and “Y” consists of about 5-10, 11-20, 21-30, 31-40, 41-50, or 5-50 nucleic acid bases; wherein X, R, Z, and Y are covalently joined, at least two nucleotides of Y are covalently linked by a non-nucleic acid linker, and at least 20%, 21%, 22%, 23%, 24%, 25%, 26%, 27%, 28%, 29%, or 30% of the total number of bases are universal or generic bases.

The oligonucleotides described herein can be sold separately or can be formulated into medicaments or pharmaceuticals. That is, embodiments of the invention include medicaments or pharmaceuticals comprising an oligonucleotide, wherein at least 20%, 21%, 22%, 23%, 24%, 25%, 26%, 27%, 28%, 29%, or 30% of the total number of bases of said oligonucleotide are universal or generic bases and may or may not contain other unnatural bases. Preferred embodiments include pharmaceuticals comprising said oligonucleotides, wherein at least 2, 3, 4, 5, 6, 7, 8, 9, or 10 of said universal and/or generic bases are juxtaposed. The section below describes the preparation of medicaments and pharmaceuticals comprising the oligonucleotides of the invention.

Pharmaceutical Embodiments

Embodiments of the invention also include methods of making and using the oligonucleotides described above, in particular methods of making and using pharmaceuticals or medicaments comprising the antisense oligonucleotides described herein. One embodiment concerns a method of designing an oligonucleotide, which involves identifying a sequence that corresponds to or complements a target sequence and substituting sufficient bases within said sequence with universal or generic bases so as to achieve an overall composition in which at least 20%, 21%, 22%, 23%, 24%, 25%, 26%, 27%, 28%, 29%, or 30% of the total number of bases are universal or generic bases. By one approach, a sequence that interacts with a target identified as being associated with a disease is selected (e.g., a selection is made from one or more of the oligonucleotides listed in U.S. Pat. Nos. 6,159,694; 6,228,642; 5,968,748; 6,133,031; and 5,840,845 and at least 20%, 21%, 22%, 23%, 24%, 25%, 26%, 27%, 28%, 29%, or 30% of the total number of bases are swapped with universal or generic bases, wherein at least two of said universal and/or generic bases are juxtaposed. Desirably, all of the universal bases are juxtaposed or are clustered at either the 5′ or 3′ end of the oligonucleotide. However, the invention is not limited to this embodiment. The introduction of blocks of universal bases was found to improve the antisense inhibition of a gene associated with cancer as compared to control treatments.

The active ingredients of the pharmaceutical embodiments of the invention (the antisense oligonucleotides) can be provided neat or with a suitable pharmaceutically acceptable carrier, e.g., sterile pyrogen-free saline solution. The active ingredients of the invention can be formulated for administration by all conventional routes including, but not limited to, parenterally, transbronchially, transdermally, topically, and orally. The formulation may be, in addition, an implant, slow release, transdermal release, sustained release, and coated with one or more macromolecules to avoid degrdation of the antisense molecule prior to reaching the selected target.

More specifically, parenteral administration, that is, subcutaneously, intravenously, intramuscularly, or interperitoneally, can be accomplished, for example, by formulating the pharmaceutical comprising the antisense molecules into injectable dosages in a physiologically acceptable diluent with a pharmaceutical carrier. Solutions for parenteral administration may be in the form of infusion solutions. A pharmaceutical carrier may be, for example, a sterile liquid or mixture of liquids such as water, saline, aqueous dextrose and related sugar solutions, an alcohol such as ethanol, glycols such as propylene glycol or polyethylene glycol, glycerol ketals such as 2,2 dimethyl 1,3 dioxolane 4 methanol, ethers such as poly(ethyleneglycol)400, oils, fatty acids, fatty acid esters or glycerides, with or without the addition of a pharmaceutically acceptable surfactant such as a soap or detergent, suspending agent such as pectin, carbomers, methylcellulose, hydroxypropylmethylcellulose, or carboxymethylcellulose, or emulsifying agent or other pharmaceutically acceptable adjuvants. Examples of oils which may be used in parenteral formulations include petroleum, animal, vegetable, or synthetic oils such as, for example, peanut oil, soybean oil, sesame oil, cottonseed oil, corn oil, olive oil, petrolatum, and mineral oil. Suitable fatty acids include, for example, oleic acid, stearic acid, and isostearic acid. Suitable fatty acid esters include ethyl oleate and isopropyl myristate. Suitable soaps include alkaline metal, ammonium and triethanolamine salts of fatty acids. Suitable detergents include cationic detergents such as dimethyl dialkyl ammonium halides and alkyl pyridinium halides; anionic detergents such as alkyl, aryl and olefin sulfonates, monoglyceride sulfates and sulfosuccinates; nonionic detergents such as fatty amine oxides, fatty acid alkanolamides and polyoxyethylenepropylene copolymers; and amphoteric detergents such as alkyl α. aminopropionates and 2 alkylimidazoline quaternary ammonium salts; as well as mixtures of detergents. Parenteral preparations will typically contain from about 0.5% to about 25% by weight of active ingredient in solution. Preservatives and buffers may also be used advantageously. Injection suspensions may include viscosity increasing substances such as, for example, sodium carboxymethylcellulose, sorbitol or dextran, and may also include stabilizers. In order to minimize irritation at the site of injection, injectable compositions may contain a non ionic surfactant having a hydrophile lipophile balance (HLB) of from about 12 to about 17. The quantity of surfactant in such formulations ranges from about 5% to about 15% by weight. The surfactant may be a single component having the above HLB or a mixture of two or more components having the desired HLB. Particular examples of useful surfactants include polyethylene sorbitan fatty acid esters, such as, for example, sorbitan monooleate.

When the present antisense molecules are administered to the respiratory system, they may be administered as a respirable formulation, more preferably in the form of an aerosol comprising respirable particles which, in turn, comprise the antisense molecules for respiration or inhalation by the subject. The respirable particles may be in gaseous, liquid or solid form, and they may, optionally, contain other therapeutic ingredients and formulation components.

When used in the lungs, the antisense molecules described herein are associated with particles of respirable size, preferably of a size sufficiently small to pass, upon inhalation, through the mouth and larynx and into the bronchi and alveoli of the lungs. In general, particles ranging from about 0.5 to 10 microns in diameter are respirable. However, other sizes may also be suitable. Particles of non-respirable size, of considerably larger diameter, which are included in the respirable formulation tend to deposit in the throat and may be swallowed. Accordingly, it is desirable to minimize the quantity of non-respirable particles in the aerosol. For nasal administration, a particle size in the range of 10-500 μm is preferred to ensure their retention in the nasal cavity.

Liquid pharmaceutical compositions comprising the antisense molecules for producing a respirable formulation, e.g., an aerosol may be prepared by combining the antisense oligonucleotide with a suitable vehicle or carrier, such as sterile pyrogen-free water and/or other known pharmaceutical or veterinarily acceptable carrier. Other therapeutic compounds may be included as well as other formulation ingredients as is known in the art.

Solid particulate compositions comprising respirable dry particles may be prepared by grinding the dry anti-sense compound with a mortar and pestle, and then passing the thus ground, e.g., micronized composition through a screen, e.g., 400 mesh screen, to break up or separate large agglomerates of particles. A solid particulate composition comprising the anti-sense compound may optionally also comprise a dispersant and other known agents, which serve to facilitate the formation of a mist or aerosol. A suitable dispersant is lactose, which may be blended with the anti-sense compound in any suitable ratio, about 1:1 w/w. Other ratios may be utilized as well, and other therapeutic and formulation agents may also be included.

The antisense molecules may also be formulated with a hydrophobic carrier capable of passing through a cell membrane (e.g., liposomes). The antisense molecules with carrier may be of any suitable structure, such as unilamellar or plurilamellar. A preferred embodiment, for example, concerns the delivery of an anti-sense oligonucleotide comprised within a liposome. Positively charged lipids such as N-[1-2, 3-dioleoyloxi) propyl]-N, N, N-trimethylammoniumethylsulfate, or “DOTAP,” are particularly preferred for such particles and vesicles. However, others are also suitable. The preparation of such lipid particles is well known. See, e.g., U.S. Pat. Nos. 4,880,635 to Janoff et al., 4,906,477 to Kurono et al., 4,911,928 to Wallach, 4,917,951 to Wallach, 4,920,016 to Allen et al., 4,921,757 to Wheatley et al., the relevant sections of all of which are herein incorporated in their entireties by reference. The active ingredients described herein may also be attached to molecules which are known to be internalized by cells. Examples of molecules used in this manner are macromolecules including transferrin, asialoglycoprotein (bound to oligonucleotides via polylysine) and streptavidin, among others.

Oral dosage forms, including capsules, pills, tablets, troches, lozenges, melts, powders, solutions, suspensions and emulsions, comprising active ingredient are also embodiments. For oral dosage forms, for example, the antisense oligonucleotides may be combined with one or more solid pharmaceutically acceptable carriers, optionally granulating the resulting mixture. Pharmaceutically acceptable adjuvants may optionally be included, such as, for example, flow regulating agents and lubricants. Suitable carriers include, for example, fillers such as sugars, cellulose preparations, calcium phosphates; and binders such as methylcellulose, hydroxymethylcellulose, and starches, such as, for example, maize starch, potato starch, rice starch, and wheat starch. Examples of orally administrable pharmaceutical preparations are dry filled capsules consisting of gelatin, and soft sealed capsules consisting of gelatin and a plasticizer such as glycerol or sorbitol. The dry filled capsules may contain the active ingredient in the form of a granulate, for example in admixture with fillers, binders, glidants, and stabilizers. In soft capsules, the active ingredient is preferably dissolved or suspended in a suitable liquid adjuvant, such as, for example, a fatty oil, paraffin oil, or liquid polyethylene glycol, optionally in the presence of stabilizers. Other oral adminstrable forms include syrups containing active ingredient, for example, in suspended form at a concentration of from about 5% to 20%, preferably about 10%, or in a similar concentration that provides a suitable single dose when administered, for example, in measures of 5 to 10 milliliters. Suitable excipients for use in oral liquid dosage forms include diluents such as water and alcohols, for example ethanol, benzyl alcohol and polyethylene alcohols, either with or without the addition of a pharmaceutically acceptable surfactant, suspending agent, or emulsifying agent. Also suitable are powdered or liquid concentrates for combining with liquids such as milk. Such concentrates may also be packed in single dose quantities.

The formulations that are contemplated are, for example, a transdermal formulation also containing carrier(s) and other agents suitable for delivery through the skin, mouth, nose, vagina, anus, eyes, ears, other body cavities, intradermally, as a sustained release formulation, intracranial, intrathecally, intravascularly, by inhalation, intrapulmonarily, into an organ, by implantation, including suppositories, cremes, gels, and the like, as is known in the art. In one particular formulation, the agent is suspended or dissolved in a solvent. In another embodiment, the carrier comprises a hydrophobic carrier, such as lipid particles or vesicles, including liposomes and micro crystals.

The medicaments comprising the antisense oligonucleotides described herein may be applied topically to treat skin symptoms and to localize to the area of the symptoms or disease. A sufficient amount of a preparation containing a compound is applied to cover the area of treatment. The compounds may be taken up in a suitable carrier for topical application such as, for example, ointments, solutions and suspensions.

Preferably, a biologically acceptable carrier is used, and more preferably a pharmaceutically or veterinarily acceptable carrier in the form of a gaseous, liquid, solid carriers, and mixtures thereof, which are suitable for the different routes of administration are used. The composition may optionally comprise other agents such as other therapeutic compounds known in the art for the treatment of the condition or disease, antioxidants, flavoring and coloring agents, fillers, volatile oils, buffering agents, dispersants, surfactants, RNA inactivating agents, antioxidants, flavoring agents, propellants and preservatives, as well as other agents known to be utilized in therapeutic compositions.

The appropriate amount of antisense nucleic acids required to inhibit expression of a gene of interest can be determined using in vitro expression analysis, protein characterization or enzymology assays, antisense inhibition studies in cell lines and animal models and in human clinical trials. The antisense molecule can be introduced into the cells expressing the protein to be inhibited by diffusion, injection, infection or transfection using procedures known in the art. For example, the antisense nucleic acids can be introduced into the body as a bare or naked oligonucleotide, oligonucleotide encapsulated in lipid, or an oligonucleotide sequence encapsidated by viral protein.

The antisense molecules are introduced onto cell samples at a number of different concentrations preferably between 1×10⁻¹⁰M to 1×10⁻⁴M. Once the minimum concentration that can adequately control gene expression is identified, the optimized dose is translated into a dosage suitable for use in vivo. For example, an inhibiting concentration in culture of 1×10⁻⁷translates into a dose of approximately 0.6 mg/kg bodyweight. Levels of oligonucleotide approaching 100 mg/kg bodyweight or higher can be possible after testing the toxicity of the oligonucleotide in laboratory animals. It is additionally contemplated that cells from a vertebrate, such as a mammal or human, are removed, treated with the antisense oligonucleotide, and reintroduced into the vertebrate.

Normal dosage amounts of pharmaceutical comprising an antisense oligonucleotide can vary from approximately 1 to 100,000 micrograms, up to a total dose of about 10 grams, depending upon the route of administration. Desirable dosages include about 250 μg-1 mg, about 50 mg-200 mg, and about 250 mg-500 mg. Pharmaceutical preparations may contain from 0.1% to 99% by weight of active ingredient. Preparations which are in single dose form, “unit dosage form”, preferably contain from 20% to 90% active ingredient, and preparations which are not in single dose form preferably contain from 5% to 20% active ingredient.

In some embodiments, the dose of a pharmaceutical comprising an antisense oligonucleotide preferably produces a tissue or blood concentration or both from approximately 0.1 μM to 500 mM. Desirable doses produce a tissue or blood concentration or both of about 1 to 800 μM. Preferable doses produce a tissue or blood concentration of greater than about 10 μM to about 500 μM. Although doses that produce a tissue concentration of greater than 800 μM are not preferred, they can be used. A constant infusion of a pharmaceutical comprising an antisense oligonucleotide can also be provided so as to maintain a stable concentration in the tissues as measured by blood levels. The total amount of active ingredient administered will generally range from about 1 milligram (mg) per kilogram (kg) of subject weight to about 100 mg/kg, and preferably from about 3 mg/kg to about 25 mg/kg. A unit dosage may contain from about 25 mg to 1 gram of active ingredient, and may be administered one or more times per day.

The exact dosage is chosen by the individual physician in view of the patient to be treated. Dosage and administration are adjusted to provide sufficient levels of the active moiety or to maintain the desired effect. Additional factors that can be taken into account include the severity of the disease, age of the organism being treated, and weight or size of the organism; diet, time and frequency of administration, drug combination(s), reaction sensitivities, and tolerance/response to therapy. Short acting pharmaceutical compositions are administered daily or more frequently whereas long acting pharmaceutical compositions are administered every 2 or more days, once a week, or once every two weeks or even less frequently.

The antisense preparation may optionally contain other therapeutic ingredients as well as other typical ingredients for a particular formulation. Examples of other agents are analgesics such as acetaminophen, anilerdine, aspirin, buprenorphine, butabital, butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx. Anti-anxiety agents are also useful including Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Anti anxiety agents associated with mental depression, such as Chlordiazepoxide, Amitriptyline, Loxapine Maprotiline and Perphenazine, among others. Anti-inflammatory agents such as non-rheumatic Aspirin, choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin, anti-inflammatories for ocular treatment such as Diclofenac, Flurbiprofen, Indomethacin, Ketorolac, Rimexolone (generally for post-operative treatment), anti-inflammatories for, non-infectious nasal applications such as Beclomethaxone, Budesonide, Dexamethasone, Flunisolide, Triamcinolone, and the like. Soporifics (anti-insomnia/sleep inducing agents) such as those utilized for treatment of insomnia, including Alprazolam, Bromazepam, Diazepam, Diphenhydramine, Doxylamine, Estazolam, Flurazepam, Halazepam, Ketazolam, Lorazepam, Nitrazepam, Prazepam Quazepam, Temazepam, Triazolam, Zolpidem and Sopiclone, among others. Sedative including Diphenhydramine, Hydroxyzine, Methortrimeprazine, Promethazine, Propofol, Melatonin, Trimeprazine, and the like. Sedatives and agents used for treat of petit mal and tremors, among other conditions, such as Amitriptyline HCl; Chlordiazepoxide, Amobarbital; Secobartital, Aprobartital, Butabarbital, Ethchiorvynol, Gluthethimide, L-Tryptophan, Mephobartital, MethoHexital Na, Midazolam Hel, Oxazepam, Pentobarbital Na, Phenobarbital, Secobarbital Na, Thiamylal Na, and many others. Agents used in the treatment of head trauma (Brain Injury/Ischemia), such as Enadoline HCl (e.g., for treatment of sever head injury; orphan status, Warner Lambert), cytoprotective agents, and agents for the treatment of menopause, monopausal symptoms (treatment), e.g., Ergotamine, Balladonna Alkaloids and Phenobarbital, for the treatment of menopausal vasomotor symptoms, e.g., Clonidine, Conjugated Estrogens and Medroxyprogesterone, Estradiol, Estradiol Cypionate, Estradiol Valerate, Estrogens, conjugated Estrogens, esterified Estrone, Estropipate, and Ethinyl Estradiol. Examples of agents for treatment of pre-menstrual syndrome (PMS) are Progesterone, Progestin, Gonadotrophic Releasing Hormone, Oral contraceptives, Danazol, Luprolide Acetate, Vitamin B6. Examples of agents for treatment of emotional/psychiatric treatments such as Tricyclic Antidepressants, including Amitriptyline CHl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRIs (selective Serotonin reuptake inhibitors), Fluoxetine HCl (Prozac), Sertaline HCl (Zoloft), Paroxetine HCl (Paxil), Fluvoxamine Maleate (Luvox), Venlafaxine CHl (Effexor), Serotonin, Serotonin Agonists (Fenfluramine), and other over the counter (OTC) medications. The section below describes some of therapeutic uses of the oligonucleotides described herein.

Therapeutic Applications

The oligonucleotides described herein are useful to treat and/or prevent animal disease, preferably human disease, and most preferably cancer. By one approach, the antisense oligonucleotides described herein, which are complementary to genes associated with cancer, are administered to a patient suffering from cancer, whereby the oligonucleotides reduce the function of the gene by antisense inhibition. A group of preferred cancer targets include transforming oncogenes, such as, ras, src, myc, and bcl-2, among others. Other examples are receptors for oncogenes, such as EGF receptor and related receptors, including but not limited to HER2/NEU, BRCA1, c-erb-b 2, and the p185 receptor. Alternatively, the action of the oncogene may be blocked by blocking the expression of a protein that is involved in the signal transduction. The expression of a protein may be blocked by targeting specific parts of the gene with antisense oligonucleotides. For example, in some cases, the initiation codon of the gene. Other targets are those to which present cancer chemotherapeutic agents are directed to, such as various enzymes, primarily, although not exclusively, thymidylate synthetase, dihydrofolate reductase, thymidine kinase, deoxycytodine kinase, ribonucleotide reductase, and the like.

In one embodiment, at least one of the mRNAs to which the antisense oligonucleotide is targeted encodes proteins such as transcription factors, stimulating and/or activating factors, intracellular and extracellular receptors, chemokines, chemokine receptors, interleukins, interleukin receptors, endogenously produced enzymes, immunoglobulins, antibody receptors, central nervous system and peripheral nervous system receptors, adhesion molecules, defensins, growth factors, vasoactive peptides and receptors, and binding proteins among others.

In a further embodiment, at least one of the mRNAs to which the antisense oligo is targeted includes but is not limited to: sympathomimetic receptors, parasympthetic receptors, GABA receptors, adenosine receptors, bradykinin receptor, insulin receptors, glucagon receptors, prostaglandin receptors, thyroid receptors androgen receptors, anabolic receptors, extrogen receptors, progesterone receptors, receptors associated with the coagulation cascade, and histamine receptors.

The following example describes in greater detail one technique that can be used to make the oligonucleotides described herein.

### EXAMPLE 1

By one approach, the oligonucleotides described herein were made using a Perkin-Elmer Applied Biosystems Expedite synthesizer. All reagents were used dry (<30 ppm water) and the oligonucleotide synthesis reagents were purchased from Glen Research. Amidites in solution were dried over Trap-paks (Perkin-Elmer Applied Biosystems, Norwalk, Conn.). A solid support previously derivatized with a dimethoxy trityl (DMT) group protected propyl linker was placed in a DNA synthesizer column compatible with a Perkin-Elmer Applied Biosystems Expedite synthesizer (1 mmol of starting propyl linker). The DMT group was removed with a deblock reagent (2.5% dichloroacetic acid in dichloromethane). The standard protocols for RNA and DNA synthesis were applied to amidites (0.1 M in dry acetonitrile). The amidites were activated with tetrazole (0.45 M in dry acetonitrile). Coupling times were typically up to 15 minutes depending on the amidite. The phosphonite intermediate was treated with an oxidizing Beaucage sulfurizing reagent. After each oxidation step, a capping step was performed, which placed an acetyl group on any remaining uncoupled 5′-OH groups by treatment with a mixture of two capping reagents: CAP A(acetic anhydride) and CAP B (n-methylimidazole in THF). The cycle was repeated a sufficient number of times with various amidites to obtain the desired sequence. After the desired sequence was obtained, the support was treated at 55° C. in concentrated ammonium hydroxide for 16 hours. The solution was concentrated on a speed vac and the residue was taken up in 100 ml aqueous 0.1 ml triethylammonium acetate. This material was then applied to an HPLC column (C-18, Kromasil, 5 mm, 4.3 mm diameter, 250 mm length) and eluted with an acetonitrile gradient (solvent A, 0.1 M TEAA; solvent B, 0.1 M TEAA and 50% acetonitrile) over 30 minutes at 1 ml/min flow rat. Fractions containing greater than 80% pure product were pooled and concentrated. The resulting residue was taken up in 80% acetic acid in water to remove the trityl group and reapplied to a reverse phase column and purified as described above. Fractions containing greater than 90% purity were pooled and concentrated.

By following the approach described above with modifications that are apparent to one of skill in the art, the oligonucleotides described herein can be made, isolated, and purified. The following example describes several preferred structures for designing the embodied oligonucleotides.

### EXAMPLE 2

Several motifs that provided greater specificity and antisense inhibition were discovered and this example describes these structures in greater detail. The oligonucleotide motifs are described using the following letter identifications:

N=Natural bases or unnatural base analogues in the oligonucleotide that hydrogen bond to natural bases in the target nucleic acid. N may be higher or lower affinity than natural bases due to base, sugar, backbone, or any other non-nucleic acid modifications or structures, (e.g. peptide nucleic acids).

S=Natural bases or unnatural base analogs or other modification that has a lower affinity to or ability to hydrogen bond to natural bases, relative to any natural base. These bases can stack in the duplex, but have lower affinity to specific opposing natural bases.

B=Any “Universal” or “generic” base analogues or other modification that can stack in duplex nucleic acid helices but do not significantly discriminate among opposing natural bases (universal, e.g. 2-deoxyinosine, 5-nitroindole, 3-nitropyrrole, 2-deoxynebularine) or that have a reduced ability to discriminate among opposing natural bases (generic, e.g. dP or dK).

X=Natural base or unnatural base substitution or any other modification within the oligonucleotide that increases the negative impact of a mismatch against the target nucleic acid. X can occur in any region of the oligonucleotide.

L=Non-nucleic acid linker (e.g. Spacer 9, Spacer 18, Spacer C3, dSpacer, all from Glen Research) either as a base substitution or contained between any pair of bases in the probe.

Representative classes of oligonucleotides for use with many of the embodiments described herein are represented below in formulae.

| | |
|---|---|
| | ( 1 )( 2 )( 3 ) |
| 1. | NNNNNNNBBBBBBNNNNNNN |
| | ( 1 )( 2 )( 3 ) |
| 2. | NNNLNNNBBBBBBNNNNNN |
| | ( 1 )( 2 )( 3 ) |
| 3. | NNNNNNNLBBBBBNNNNNNN |
| | ( 1 )( 2 )( 3 ) |
| 4. | NNNNNNLBBBBBBLNNNNNN |
| | ( 1 )( 2 )( 3 ) |
| 5. | NNNNNNNBBBBBBNNNLNN |
| | (1 )(2)(3)(4)( 5 ) |
| 6. | NNNNNBBBNNNBBBNNNNN |

In many cases, the desired target nucleic acid contains only a single mutation (e.g., a single nucleotide polymorphism or SNP) and one must be able to selectively inhibit the mutant nucleic acid but not impair the ability of the wild-type nucleic acid to encode protein. Aspects of the invention have been developed that allow for this level of sensitive detection. TABLE 1 describes the unnatural and natural base choices that allow one to: 1) discriminate SNP bases more precisely that natural bases alone, and 2) create the higher and lower affinity blocks included in the oligonucleotides of the preferred embodiment.

TABLE 1

Natural Base to Avoid Binding

| | G | A | T | C |
|---|---|---|---|---|
| Natura Base to Bind in the Target | | | | |
| G | — | *N4.EtdC | dC | dC |
| | — | ^{##}not 5-Me-dC | 5-Me-dC | 5-Me-dC |
| | — | ^{##}not dC | | |
| A | 2-Thio-dT | — | | |
| | | — | 2-Thio- | 2-Thio-dT |
| | not dT | — | dT | |
| T | **2-amino-dA | | — | 2-amino-P |
| | | 2-amino-dA | — | ^{#}not 2- |
| | not dA | | — | amino-dA |
| | | | | ^{#}not dA |
| C | dG | ***dX | dX | — |
| | dG | not dG | not dG | — |

Relative binding strength estimates contributing to choices:
*5-Me-dC:dG>dC:dG>N4-Et-dC:dG?>?N4-Et-dC:dA
^{##}dT:dA=5-Me-dC:dA>dC:dA=dU:dA
**2-amino-dA:dT>dA:dT>>2-amino-dA:dG
***dX:dC=dA:dU>>dX:dG<dA:dG=dA:dI
^{#}2-amino-dA:dC>dA:dC=dA:dU>>dA:dI

It is further contemplated that placing an unnatural base that has a modified affinity, preferably a lower affinity, but a higher affinity may also be used, increases specificity and concomitantly antisense inhibition.

The table shown above is designed to exemplify the way any natural or unnatural base or analogue can be selected to maximize SNP discrimination in combination with universal or generic bases. Given any of the general structure permutations shown above (numbered 1-6), for any SNP in any position, Table 1 allows one to determine which base to discriminate and target the specific SNP base. For example, it can be used to determine which base one wants this probe to bind to in the target versus the SNP base in the non-target. Most wild-type versus mutant SNP detection systems have both wild-type and mutant targets in the mixture, so one has to absolutely maximize the ability to discriminate the two SNP bases that define wild-type versus mutant and the Table allows one to do so. If one were trying to get better discrimination between an adenine in the wt target and guanine in the mutant target (the SNP), one could go to the table and look up “adenine” as the natural base and under the heading “guanine”, one finds “2-Thio-dT” which tells you that you will get the best discrimination between “A” and “G” if “2-Thio-dT” is used in the primer.

The next example illustrates that the incorporation of universal or generic bases in an oligonucleotide facilitates the differentiation of two sequences that differ by a single nucleotide.

### EXAMPLE 3

In these experiments it was demonstrated that oligonucleotides having universal bases facilitate the identification of a single nucleotide base change in a nucleic acid. In a first set of experiments, the differences in melting behaviors of a natural probe/target complex and an oligonucleotide probe having 5 juxtaposed universal bases/target complex was ascertained. Multiple melting temperature determinations were performed for each probe/target combination. All mixtures were heated to 85-95° C. for 10-15 minutes and allowed to cool to room temperature before use. Melting temperatures were determined by UV absorbence in sealed quartz cuvettes using a Varian Cary 3E UV-Visible Spectrophotometer with a Varian Cary temperature controller, controlled with Cary 01.01(4) Thermal software. Temperature gradients decreased from 85° C. to 25° C. at 1° C. per minute.

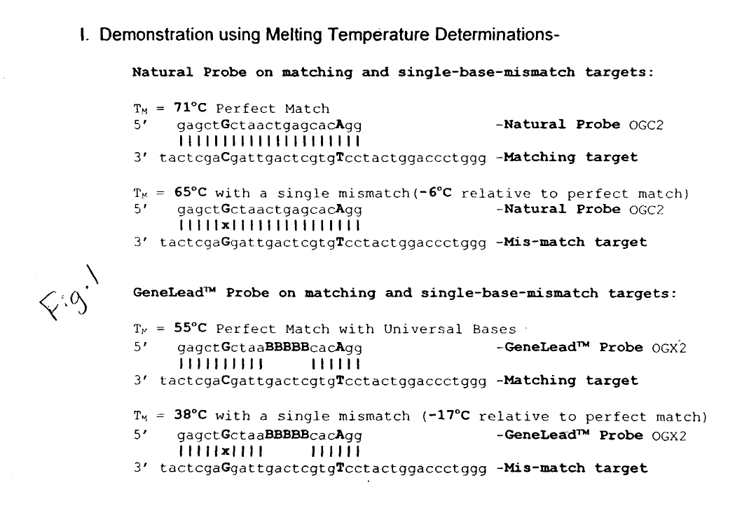

The mutant target contained a single mismatch, a G—G mismatch to both probes, OGC2 and OGX2. As shown in FIG. 1, the all-natural probe OGC2 (SEQ ID NO: 2) bound to the mismatch target #1090 (SEQ ID NO: 8) with a differential melting temperature of −6° C. relative to the perfect match wild-type target #1088 (SEQ ID NO: 7). OGX2 (SEQ ID NO: 4), the oligonucleotide containing 5 universal bases, bound with a differential melting temperature of −17° C. relative to the perfect match. In the presence of five juxtaposed universal bases, therefore, the single purine-purine mismatch decreases the perfect-probe-to-target melting temperature by 17° C., thereby facilitating the detection of the SNP. This demonstrates that the improvements herein can be used to develop very specific antisense oligonucleotides.

The following example details experiments that examined the effect of salt concentration on the oligonucleotides described herein.

### EXAMPLE 4

Melting temperatures were determined for the following three probes containing generic and universal bases in various salt concentrations and the results were compared to those obtained using a control probe without the generic and universal bases (5′ natural OGC2). The probes analyzed included 5′ OGX1 (SEQ ID NO: 3), 5′OGX3 (SEQ ID NO: 5), 5′OGX5 (SEQ ID NO: 6) and 5′natural OGC2 (SEQ ID NO: 2). The target was #1088 (SEQ ID NO: 7). Oligonucleotide probes and DNA targets were at 0.35 to 0.40 O.D. each per milliliter in both an enzymatically relevant buffer system (KCl/Mg++) or in a non-physiological, high salt buffer system (NaCl/PO₄₋₋):

| | |
|---|---|
| KCl/Mg++ Buffer: | NaCl/PO₄−− Buffer: |

| | | | |
|---|---|---|---|
| 20 | mM Tris-HCl, pH = 7.5 | 10 | mM NaH₂PO₄, pH = 7.0 |
| | at 20° C. | | at 20° C. |
| 100 | mM KCl | 1 | M NaCl |
| 10 | mM MgCl₂ | 0.1 | EDTA |
| 0.05 | mM DTT | | |
| 2.5% | w/v sucrose | | |

Multiple melting temperature determinations were performed for each probe/target combination. All mixtures were heated to 85-95° C. for 10-15 minutes and allowed to cool to room temperature before use. Melting temperatures were determined by UV absorbence in sealed quartz cuvettes using a Varian Cary 3E UV-Visible Spectrophotometer with a Varian Cary temperature controller, controlled with Cary 01.01(4) Thermal software. Temperature gradients decreased from 85° C. to 25° C. at 1° C. per minute.

As shown in TABLE 2, the difference in melting behavior of oligonucleotides having universal or generic bases and natural oligonucleotides were not influenced by salt concentration.

TABLE 2

| | KCl/Mg++ | | NaCl/PO₄−− | |
|---|---|---|---|---|
| | Match | MisMatch | Match | MisMatch |
| Probe | T_{M} | T_{M} | T_{M} | T_{M} |
| 5′ OGX1 | <25 | 53 | <25 | 58 |
| 5′ OGX3 | <25 | 51 | <25 | 57 |
| 5′ OGX5 | <25 | 56 | <25 | 63 |
| 5′ natural OGC2 | 64 | 70 | 71 | 75 |

The following example provides more evidence that the incorporation of at least two juxtaposed universal bases in an antisense oligonucleotide provides an improved sensitivity and concomitantly better antisense inhibition.

### EXAMPLE 5

The melting behavior of control probes (i.e., no universal and generic bases) OGC1 (SEQ ID NO: 1) and OGC2 (SEQ ID NO: 2) annealed to two different target DNA's:#1088 (SEQ ID NO: 7), which contains a G to C match, and #1090 (SEQ ID NO: 8), which contains a G—G mismatch, were compared to the melting behaviors of probes containing universal and generic bases. The universal or generic base containing probes analyzed included 5′ OGX1 (SEQ ID NO: 3), 5′OGX2 (SEQ ID NO: 4), and 5′ OGX5 (SEQ ID NO: 6).

A polyacrylamide gel bandshift experiment was then conducted as follows. The gel matrix was 20% acrylamide (19:1 acrylamide to bis-acrylamide) in 1×TBE buffer and “extra” salts: 20 mM Tris-HCl, pH=7.5 at 20° C., 100 mM KCl, 10 mM MgCl₂, 0.05 mM DTT, 2.5% w/v sucrose. Oligonucleotide mixtures were at approximately 5 micromolar each in formamide/dye sample buffer plus 2× of the extra salt concentrations in the acrylamide gel mixture. The gel was run in 1×TBE at 93V (19 mA) and the buffer and gel temperatures were kept stable at 26° C. during the entire electrophoretic run.

The polyacrylamide gel was scanned, lanes 1-12, and the oligonucleotide probe/DNA target sequences were analyzed. Probe and DNA target designations are provided in TABLE 3. Lanes 11 and 12 of the gel marked the position of unbound target DNAs (#1088, perfect match and #1090, single base mismatch, respectively).

Lanes 1, 2, 3, and 4 of the gel showed that the all-natural-base probes (OGC1 and OGC2) could not distinguish the single base mismatch target (#1090, lanes 2 and 4) from the perfectly matched target (#1088, lanes 1 and 3). Lanes 5 through 10, on the other hand, graphically revealed the ability of the probes containing juxtaposed universal bases to detect a single-base-mismatch under these conditions. Thus, the results above provide more evidence that antisense oligonucleotides comprising juxtaposed universal bases are more specific for a target than conventional oligonucleotides, which translates into improved antisense inhibition.

TABLE 3

| | | |
|---|---|---|
| Size | Name | Identity |

| | | | |
|---|---|---|---|
| Control Oligonucleotides: | | | |
| 5′ ctGctaactgagcacAggatg (C6-NH2) | 21 mer | OGC1 | control |
| | | (SEQ ID NO:1) | |
| 5′ gagctGctaactgagcacAgg (C6-NH2) | 21 mer | OGC2 | control |
| | | (SEQ ID NO:2) | |
| Experimental Oligonucleotides | | | |
| 5′ ctGctaBBBBBgcacAggatg (C6-NH2) | 21 mer | OGX1 | 6/5/10 |
| | | (SEQ ID NO:3) | |
| 5′ gagctGctaaBBBBBcacAgg(C6-NH2) | 21 mer | OGX2 | 10/5/6 |
| | | SEQ ID NO:4 | |
| 5′ gctGctaBBBBBgcacAgg (C6-NH2) | 19 mer | OGX3 | |
| | | SEQ ID NO:5 | |
| 5′ gagctGctBBBBBagcacAgg(C6-NH2) | 21 mer | OGX5 | 8/5/8 |
| | | SEQ ID NO:6 | |
| Target DNA's | | | |
| 3′ tactcgaCgattgactcgtgTcctactggaccctggg | #1088 | Target | 37 mer |
| | | (SEQ ID NO:7) | |
| 3′ tactcgaGgattgactcgtgTcctactggaccctggg | #1090 | Target | 37 mer |
| | | (SEQ ID NO:8) | |

The next example describes the use of the oligonucleotides described herein to inhibit the human Bcl2 gene so as to treat or prevent many types of cancer.

### EXAMPLE 6

B cell lymphoma-associated gene 2 (Bcl2) is a “normal” human gene that is overexpressed in a majority of human cancer types. The Bcl2 protein regulates cell death and BCl overexpression is known to cause cells to be chemotherapy and radiation resistant. The following Bcl2-targeted antisense molecule is synthesized:

Oligomers: The following BCL2-targeted antisense molecules were synthesized:

| | | |
|---|---|---|
| 1060 BCL2 18-base antisense | 5′TCTCCCAGCGTGCGCCAT | (SEQ ID NO:9) |
| 1061 BCL2 4 mismatch control | 5′TCTACCCGCGTCCGGCAT | (SEQ ID NO:10) |
| 1062 BCL2 Cleaver | 5′TCTCCCAGCGTG9GAGUACUCAACCAGC1 | (SEQ ID NO:11) |
| 1063 BCL2 Cleaver | 5′TCTCCCAGCGBB9GAGUACUCAACCAGC1 | (SEQ ID NO:12) |
| 1066 BCL2 Anchor | 5′GCUGGUUGAGUACUC9cgccat1 | (SEQ ID NO:13) |

where NNNN=phosphorothioate deoxyribonucleic acid (PS DNA), NNNN=2′-O-methyl ribonucleic acid (2′-OMe RNA), nnnn =2′-O-Methyl phosphorothioate ribonucleic acid (2′-OMe PS RNA), and NNNN=C-5 Propynyl-modified phosphorothioate deoxyribonucleic acid (Propynyl), 9 =Glen Research linker #9, 1 =Glen Research propyl linker on CPG (Cat. No. **), F=Molecular Probes Fluorescein (Cat. No. F-1907), and R=Molecular Probes Rhodamine (Cat. No. X-491).

1062 (a 12-mer, RNase H-substrate cleaver) and 1063 (a 12-mer, RNase H-substrate cleaver with a 6-base C-5 propynyl-modified “tack” at the 5′ end of the RNase H-substrate region) both hybridized to 1066 (a 6-mer, non-RNase H-substrate anchor) to create active antisense constructions against BCL2.

1060 (based on a published oligonucleotide known clinically as G3139) is a conventional 18-mer all-phosphorothioate antisense oligonucleotide. 1060 hybridizes to the BCL2 pre-mRNA across the first 6 codons of the open reading frame.

1061 is a conventional all-phosphorothioate 18-mer, 4 base mismatch control to the BCL2 gene.

Tissue Culture: The cell line that was used for this demonstration was T-24 (American Type Culture Collection #HTB-4), a human bladder carcinoma line known to over express BCL2.

T-24 was maintained in culture using standard methods at 37° C., 5% CO₂, in 75-cm² flasks (Falcon, Cat. No. 3084) in McCoy's 5A medium (Mediatech, Cat. No. 10-050-CV) with 10% serum (Gemini Bio-Products, Cat. No. 100-107) and penicillin-streptomycin (50 IU/mL, 50 mcg/mL, Mediatech, Cat. No. 30-001-LI).

For antisense experiments T-24 were plated into 12-well plates (Falcon, Cat. No. 3043) at 75,000 cells/well and allowed to adhere and recover overnight before oligo-nucleotide transfections began.

Transfection of Oligonucleotides into T-24 cells: Oligonucleotides were transfected into T-24 cells with a cationic lipid-containing cytofectin agent LipofectACE™ (GibcoBRL, Cat. No. 18301-010). LipofectACE has been shown to give efficient nuclear delivery of fluorescently labeled antisense constructions in T-24.

Antisense and conventional all-phosphorothioate oligonucleotides were diluted into 1.5 mL of reduced serum medium Opti-MEM© I (GibcoBRL, Cat. No. 11058-021) to a concentration of 400 nM each. The oligonucleotide-containing solutions were then mixed with an equal volume of Opti-MEM I containing LipofectACE sufficient to give a final lipid to oligonucleotide ratio of 5 to 1 by weight.

The final concentration of oligonucleotide was 200 nM. The oligonucleotide/lipid complexes were incubated at room temperature for 20 minutes before adding to tissue culture cells.

Cells were washed once in phosphate buffered saline (PBS, Mediatech Cat. No. 21-030-LV) to rinse away serum-containing medium and then one mL of transfection mix was placed into each well of a 12-well plate. All transfections were performed in triplicate.

The cells were allowed to take up oligonucleotide/lipid complexes for 24 hours prior to harvesting of total cellular RNA. Mock transfections consisted of cells treated with Opti-MEM I only.

Total Cytoplasmic RNA Isolation: After 22 hours of antisense treatment, total RNA was harvested from the cells. The cells were released from the plates by trypsinizing (Tryspin/EDTA, Mediatech Cat. No. 25-052-LI) according to standard methods. The triplicate groups of cells were pooled and total cytoplasmic RNA was isolated according to the RNeasy Protocol and spin columns from an RNeasy Kit (QIAGEN, Cat. No. 74104).

The RNA was DNase I treated and UV quantitated according to standard methods

Polymerase Chain Reactions to Detect BCL2 RNA: Reverse Transcriptase/Polymerase Chain Reactions (RT-PCR) were performed with the methods and materials from a SuperScript One-Step RT-PCR Kit from GibcoBRL (Cat. No. 10928-026). The RT-PCR reactions to detect BCL2 were performed with BCL2-specific primers from the literature: upstream 5′ ggtgccacctgtggtccacctg and downstream 5′ cttcacttgtggcccagatagg (both primers were normal DNA) and 1 μg of input total RNA. Control RT-PCR reactions against β-actin were also performed with primers from the literature: upstream 5′ gagctgcgtgtggcccgagg (SEQ ID NO: 14) and downstream 5′ cgcaggatggcatggggggcatacccc SEQ ID NO: 15) (both primers were normal DNA) and 0.1 g of input total RNA.

All BCL2 and β-actin RT-PCR reactions were performed according to the following program on a PTC-100 thermocycler (MJResearch): Step 1, 50° C. for 35 minutes; Step 2, 94° C. for 2 minutes; Step 3, 60° C. for 30 seconds; Step 4, 72° C. for 1 minute; Step 5, 94° C. for 30 seconds; Step 6, Go to Step 3, 35 more times; Step 7, 72° C. for 10 minutes; Step 8, End.

All RT-PCR products were separated on a 4% Super Resolution Agarose TBE gel (Apex Cat. No. 20-105) and stained with SyberGold (Molecular Probes, Cat. No. S-11494), according to the manufacture's instructions. Gels were photographed on Polaroid Type 667 film

TABLE 4
Reduced Target Gene Expression (BCL2) Confirms
that Antisense Constructions With Universal
Bases Are Active and Specific in Cells

| | | | | | BCL2 | β-actin |
|---|---|---|---|---|---|---|
| | | Cleaver | Anchor | All-PS | mRNA | mRNA |
| Lane | Treatment | Oligo | Oligo | Oligo | level | level |
| 1 | Mock | — | — | — | ++++ | ++++ |
| 2 | Conventional | | — | 1060 | + | ++++ |
| | antisense | | | | | |
| 3 | Conventional | — | — | 1061 | ++++ | ++++ |
| | control | | | | | |
| 4 | Cleaver | 1062 | — | | ++++ | ++++ |
| | alone | | | | | |
| 5 | Antisense | 1062 | 1066 | — | + | ++++ |
| | assembled | | | | | |
| 6 | Cleaver | 1063 | — | — | +++ | ++++ |
| | alone | | | | | |
| 7 | Antisense | 1063 | 1066 | — | + | ++++ |
| | assembled | | | | | |
| 8 | Anchor | — | 1066 | — | ++++ | ++++ |
| | alone | | | | | |

Results

The antisense anti-BCL2 constructions dropped BCL2 RNA levels significantly compared to control treatments. Compare lanes 5 (oligos 1062+1066) and 7 (1063+1066) to lanes 1 (mock treatment) and 3 (conventional antisense control).

None of the oligonucleotides and antisense constructions showed any activity against the control gene β-actin.

This is significant because it clearly demonstrates antisense activity with: (a) only a 6 base anchor (1066, lanes 5 and 7), (b) two nitroindole universal bases, “B”, replacing natural bases in the cleaver sequence (1063 alone, and 1063+1066, lanes 6 and 7), and (c) that antisense activity is general and could be easily observed against another human target genes.

The experimental result that an anchor as short a 6 bases long combined with a cleaver containing nitroindole as a universal base (1063+1066) could form a antisense construct with effective antisense activity inside cells clearly confirmed the validity of our cell-free work with SEAP-targeted antisense oligonucleotides. It should be understood that although the example above was performed with a coupled two component oligonucleotide (antisense assembled) a single antisense oligonucleotide containing the same domains would be expected to perform at least as well. The data above demonstrates that improved antisense oligonucleotides containing juxtaposed universal bases can be developed and that these oligonucleotides are effective antisense inhibitors of the BCL2 gene and, thus, inhibitors of the proliferation of cancer cells. Oligonucleotides comprising the sequence and modifications above can be incorporated into pharmaceuticals and adminstered to a subject suffering from cancer so as to inhibit the proliferation of cancer cells and prevent further spread of the disease. The next example describes the use of antisense oligonucleotides that complement five different genes expressed in melanoma cells so as to inhibit the proliferation of cancer cells.,

### EXAMPLE 7

This example describes experiments that were conducted to verify that antisense oligonucleotides comprising a plurality of juxtaposed universal bases could be used to inhibit genes expressed in melanoma cells. Accordingly, a series of antisense oligonucleotides containing modified bases and blocks and mixed blocks of ambiguous, degenerate, and universal bases were synthesized according to standard methods. Each oligonucleotide was fluorescently labeled and evaluated in A549 cells for intranuclear uptake and biological activity as described below.

Antisense oligonucleotide sequences were chosen based on the position in the target gene, base composition, known positive antisense effects and known oligonucleotide artifacts. Oligonucleotide transfection methods to achieve intranuclear delivery were established using fluorescent oligonucleotides and direct observation of cell nuclei. Once intranuclear delivery was confirmed, antisense oligonucleotides were evaluated for antisense activity, toxicity and specificity by RT-PCR reactions.

Oligonucleotide Delivery Evaluations were performed as follows: Fluorescently labeled FAM-G3139 (F-G3139, JBL Scientific) and FAM-Oasis1039 (synthesized by TriLink Biotechnologies) were resuspended at 200 μM in TE buffer, pH=7.5 (Maniatis). For transfection assays, F-G3139 or FAM-Oasis1039+Oasis1017 were diluted to 250 nM final oligonucleotide or complex in OptiMEM I (Life Technologies, Cat. No. 11058-021) and mixed with cationic vehicles (see Table 1) at a 2:1 to 6:1 ratio by weight.

Cells were plated on glass chamber slides at 60 to 90% confluence (Nunc, Cat. No. 154534) and allowed to grow to 70-100% confluence were treated with transfection mixtures overnight and then formaldehyde-fixed and mounted using standard methods (Maniatis). Nuclear accumulation of fluorescein-labeled oligonucleotide was evaluated under UV illumination at 100-400×magnification, using a Nikon Labophot 2 microscope with PlanApo objectives (Nikon). Bright intranuclear fluorescence was indicative of productive oligonucleotide delivery. Optimizations of several initially active lipids was performed to identify the best delivery vehicles, such as CellFECTIN (Gibco/BRL Cat. No. 10362-010) at a 2:1 lipid/DNA ratio by weight for the human melanoma cell line A549.

Oligonucleotide transfections for biological activity were performed in A549 cells, a human melanoma cell line cultured under standard conditions (5% carbon dioxide, 37° C.). A459 cells were transfected efficiently with Cellfectin and the modified oligonucleotides and conventional all-phosphorothioate oligonucleotides.

A series of antisense oligonucleotides containing modified bases and blocks and mixed blocks of ambiguous, degenerate, and universal bases were synthesized according to standard methods. Each oligonucleotide was fluorescently labeled and evaluated in A549 cells for intranuclear uptake and biological activity as described herein.

A549 cells were plated and allowed to grow and recover to an initial density of 70-80% before being transfected with oligonucleotides for biological activity determinations. Each oligonucleotide was transfected in one well of a 6 well plate (Falcon, Cat. No. 3046) using 2.5 mL/well transfection mix. All transfections were incubated for 20-24 hours at 5% CO2, 100% humidity, 37° C. Cells were washed with phosphate buffered saline (Cellgro, Cat. No. 21-030-LV) immediately before total RNA isolations.

Final transfection mixes were 200 nM oligonucleotides. Transfection reactions were prepared by combining equal volumes of 2×oligonucleotide in OptiMEM I and 2×lipid in OptiMEM I to give the final 1×concentration. Transfection mixtures were incubated for 15 minutes at room temperature before placing on cells. Cells were transfected for up to 24 before the isolation of total RNA.

Total RNA was isolated as follows: Total RNA samples were prepared at room temperature using a guanidinium hydrochloride-denaturation/ silica gel column-based method (RNeasy® Mini Kit, QIAGEN, Cat. No. 74104) exactly according to the manufacture's recommendations and methods for the isolation of total cytoplasmic RNA. Total RNA was treated with DNase I on the column to remove any contaminating genomic DNA according to the manufacturer's recommendations and methods (RNase-Free DNase kit, QIAGEN, Cat. No. 79254). After column elution, RNA samples were ethanol precipitated and washed (all according to Maniatis et al.) and resuspended in ultra pure RNase-free water (QIAGEN) for reverse transcription-polymerase chain reactions (RT-PCR).

RT-PCR was performed on the Total RNA as follows: Total RNA was isolated from 6-well tissue culture plates using QIAGEN's RNeasy Mini Kit and the recommended methods. RT-PCRs were performed in an MJ Research PTC-100 Thermocycler with Hot Bonnet, using SUPERSCRIPT One-Step RT-PCR with Platinum Taq kit reagents and protocol (Life Technologies, cat. no. 10928-042). All reactions were 50 μL final volume with 0.2 μM of each primer. Input total RNA (ng) for each gene and the RNA sources given above.

TABLE 5

RT-PCR Primers

| | | | | | |
|---|---|---|---|---|---|
| | | Pos. | | | Amp. |
| Name | Gene | ** | Primer Sequence | *Tm | Size |

| | | | | | | |
|---|---|---|---|---|---|---|
| 1094 | STLK4 | 1411 | ctc agg tct ccc cga gtg aa | (SEQ ID NO:16) | 55.8 | 355 bp |
| 1095 | | 1747 | Cga cca ggc cag cag aaa t | (SEQ ID NO:17) | | |
| 1100 | PTPα | 1670 | gcg gat gat ctg gga aca aa | (SEQ ID NO:18) | 57.9 | 400 bp |
| 1101 | | 2050 | cat ggc atc aat gac gac aa | (SEQ ID NO:19) | | |
| 1104 | ZC1 | 365 | cca aag gga aca cac tca aa | (SEQ ID NO:20) | 54.8 | 305 bp |
| 1105 | | 650 | aat gcc aca aga cca aag at | (SEQ ID NO:21) | | |
| BC2 | GSK3β | | cgt gac cag tgt tgc tga gt | (SEQ ID NO:22) | 55.5 | 378 bp |
| BC3 | | | tct gct ggs agt ata cac caa | (SEQ ID NO:23) | | |
| 1098 | HRI | 914 | cac ccc aga aaa aga aaa ac | (SEQ ID NO:24) | 54.6 | 399 bp |
| 1099 | | 1293 | ttg gcc ata aca taa gga ca | (SEQ ID NO:25) | | |

After RT-PCR completion, 10 μL of 6×Type II agarose sample buffer Maniatis et. al.) were added to the tubes before running 8 μL of each reaction on 3% high resolution agarose in TBE. Gels were stained with SYBRGold (Molecular Probes) and photographed on Polaroid Type 667 film. The images were then scanned and converted to negatives. The RT-PCR Program was as follows:

| Step | Temp | Time |
|---|---|---|
| 1 | 50° C. | 0:35:00 |
| 2 | 94 | 0:02:00 |
| 3 | X* | 0:00:45 |
| 4 | 72 | 0:01:00 |
| 5 | 94 | 0:00:30 |
| 6 | Go to step | |
| | three 29 times | |
| | (30 cycles total) | |
| 7 | 72 | 0:10:00 |
| 8 | End | |

TABLE 6

Modified Antisense Oligonucleotides Against Disease Associated Genes

| | | |
|---|---|---|
| Name | Sequence | Gene |

| | | | |
|---|---|---|---|
| 3167 | (ps)(#Z# T## Z#T CEE) 2′OMe(AGC CUC CA)-FAM | (SEQ ID NO:26) | STLK4 |
| 3168 | (ps)(### TTG ZTZ TEE) 2′OMe(CGG UGU AU)-FAM | (SEQ ID NO:27) | ZC1 |
| 3169 | (ps)(#Z# ##G ZZT ZEE) 2′OMe(CCC AUA GG)-FAM | (SEQ ID NO:28) | PTP-α |
| 3170 | (ps)(#TG T## Z#G GEE) 2′OMe(UCC AGU AU)-FAM | (SEQ ID NO:29) | GSK-3β |
| 3171 | (ps)(#TG G## Z#T GEE) 2′OMe(UCA AGU CU)-FAM | (SEQ ID NO:30) | GSK-3β |
| 3172 | ps(#Z# T## Z#T #DD) 2′OMe(AGC CUC CA)-FAM | (SEQ ID NO:31) | STLK4 |
| 3173 | ps(### TTG ZTZ TDD) 2′OMe(CGG UGU AU)-FAM | (SEQ ID NO:32) | ZC1 |
| 3174 | ps(#Z# ##G ZZT ZDD) 2′OMe(CCC AUA GG)-FAM | (SEQ ID NO:33) | PTP-α |
| 3175 | ps(#TG T## Z#G GDD) 2′OMe(UCC AGU AU)-FAM | (SEQ ID NO:34) | GSK-3β |
| 3176 | ps(#TG G## Z#T GDD) 2′OMe(UCA AGU CU)-FAM | (SEQ ID NO:35) | GSK-3β mm |

D = dSpacer (Glen Research Cat. No. 10-1914)
Z = 2,6-diaminopurine (Glen Research Cat. No. 10-1085)
E = 2′-deoxynebularine (Glen Research Cat. No. 10-1041)
#=5-Methyl-deoxycytosine (Glen Research Cat. No. 10-1060)

All oligonucleotides described in Table 6 were found to have antisense activity comensurate with that of natural oligonucleotides. Table 7, lists more modified oligonucleotides that were tested.

TABLE 7

Modified Antisense Oligonucleotides Containing Blocks and Mixed Blocks of Un-
natural Bases Show Biological Activity

| | | Intra- | |
|---|---|---|---|
| | | Nuclear | Antisense |
| Oligo | Oligonucleotide Sequence and Composition | Uptake | Activity |
| 1241F1 | (6-FAM)ps[G U*C*C*A C* GGTCTC] (*) 2′OMe[CAGUAU] | + | + |
| | (SEQ ID NO:36) | | |
| 1241F2 | (6-FAM)ps[G U*C*C*A C* BBBBBB] (*) 2′OMe[CAGUAU] | + | + |
| | (SEQ ID NO:37) | | |
| 1241F3 | (6-FAM)ps[G U*C*C*A C* BBB] (*) BBB 2′OMe[CAGUAU] | + | − |
| | (SEQ ID NO:38) | | |
| 1241F4 | (6-FAM)ps[G T # # A # BBBBBB] (*) 2′OMe[CAGUAU] | + | − |
| | (SEQ ID NO:39) | | |
| 1241F5 | (6-FAM)ps[G U*C*C*A C* EEEEEE] (*) 2′OMe[CAGUAU] | + | + |
| | (SEQ ID NO:40) | | |
| 1241F6 | (6-FAM)ps[G U*C*C*A C* MMMMMM] (*) 2′OMe[CAGUAU] | − | − |
| | (SEQ ID NO:41) | | |
| 1241F7 | (6-FAM)ps[G U*C*C*A C* BEEBEE] (*) 2″OMe[CAGUAU] | ND | ND |
| | (SEQ ID NO:42) | | |
| 1241F9 | (6-FAM)ps[G U*C*C*A C* BIIBII] (*) 2′OMe[CAGUAU] | + | + |
| | (SEQ ID NO:43) | | |
| 1241F10 | (6-FAM)ps[G U*C*C*A C* KKPPPP] (*) 2′OMe[CAGUAU] | + | + |
| | (SEQ ID NO:44) | | |
| 1241F11 | (6-FAM)ps[G T # # Z # KKPPPP] (*) 2′OMe[CAGUAU] | + | + |
| | (SEQ ID NO:45) | | |
| 1241F12 | (6-FAM)ps[G T # # Z # EEEEEE] (*) 2′OMe[CAGUAU] | + | + |
| | (SEQ ID NO:46) | | |

B = 5-nitroindole (Glen Research Cat. No. 10-1044)
C* = C5-propyne-deoxycytosine (Glen Research Cat. No. 10-1014)
E = 2′-deoxynebularine (Glen Research Cat. No. 10-1041)
K = dK-CE (Glen Research Cat. No. 10-1048)
I = deoxyinosine (Glen Research Cat. No. 10-1040)
M = 3-nitropyrrole (Glen Research Cat. No. 10-1043)
P = dP-CE (Glen Research Cat. No. 10-1047)
U* = C5-propyne-deoxyuridine (Glen Research Cat. No. 10-1054)
Z = 2,6-diaminopurine (Glen Research Cat. No. 10-1085)
# = 5-Methyl deoxy C (Glen Research Cat. No. 10-1060)
(*) = Spacer9 (Glen Research Cat. No. 10-1909)
ps = phosphorothioate DNA
2′OMe = 2′-O-methyl RNA
6-FAM = FAM (fluorescein) label

Many of the oligonucleotides described in Table 7 were also found to provide significant antisense activity toward the desired target. The data above demonstrates that oligonucleotides comprising a plurality of juxtaposed universal bases significantly inhibit a plurality of genes expressed in a melanoma cell line. Similar data has been obtained in cell lines from other human cancers. These antisense oligonucleotides can be incorporated into pharmaceuticals and administered to a subject in need, as described herein, in an approach to inhibit the proliferation of melanoma cells and/or methods to treat or prevent melanoma in an afflicted subject. The next example describes the use of oligonucleotides prepared according to the teaching described herein for the treatment and prevention of diseases associated with the expression of STAT-3, such as inflammation and various forms of cancer.

### EXAMPLE 8

STAT-3 encodes a DNA-binding protein that plays a dual role in signal transduction and activation of transcription. Overexpression of STAT-3 is involved in inflammatory diseases and cancer. Others have disclosed antisense techniques to inhibit STAT-3 activity and thereby treat and/or prevent STAT-3-associated disease (See e.g., U.S. Pat. No. 6,159,694, herein incorporated by reference in its entirety).

The approach above can be improved by implementing the antisense oligonucleotide technology described herein. Accordingly, oligonucleotide sequences complementary to STAT-3 are selected based upon their efficacy at down-regulating STAT-3. Modifications are made to said oligonucleotides by incorporating blocks of at least two juxtaposed universal bases. The following oligonucleotides are used in this experiment:

Unmodified:

| | |
|---|---|
| Unmodified: | |
| GTCTGCGCCGCCGCCCCGAA | (SEQ ID NO:47) |
| GGCCGAAGGGCCTCTCCGAG | (SEQ ID NO:48) |
| TCCTGTTTCTCCGGCAGAGG | (SEQ ID NO:49) |
| CATCCTGTTTCTCCGGCAGA | (SEQ ID NO:50) |
| Modified: | |
| GTBBGCGCCGCCGCCCCGAA | (SEQ ID NO:51) |
| GGCCGAABBBCCTCTCCGAG | (SEQ ID NO:52) |
| TCCTGTTTCTCCGBBBBAGG | (SEQ ID NO:53) |
| CATCCTBBBBBBBCGGCAGA | (SEQ ID NO:54) |

Modified:

The antisense oligonucleotides are designed to target mouse STAT3. Target sequence data are from the STAT3 cDNA sequence submitted by Zhong, Z.; Genbank accession number U06922 The above chosen oligonucleotides are compared in vitro as follows: The B lymphoma cell line, BCL1 is obtained from ATCC (Rockville, Md.) BCL1 cells are cultured in RPMI 1640 medium. BCL1 cells (5×10⁶ cells in PBS) are transfected with oligonucleotides by clectroporation, at 200V, 1000 μF using a BTX Electro Cell Manipulator 600 (Genetronics, San Diego, Calif.). For an initial screen, BCL1 are electroporated with 10 μM oligonucleotide and RNA collected 24 hours later. Controls without oligonucleotide are subjected to the same electroporation conditions.

Total cellular RNA is isolated using the RNEASY® kit (Qiagen, Santa Clarita, Calif.). RNase protection experiments are conducted using RIBOQUANT™ kits and template sets according to the manufacturer's instructions (Pharmingen, San Diego, Calif.). Northern blotting is performed as described in Chiang, M -Y. et al. (J. Biol. Chem., 1991, 266, 18162-18171), using a rat cDNA probe prepared by Xho I/Sal I restriction digest of psvsport-1 plasmid (ATCC, Rockville, Md.). mRNA levels are quantitated using a Phosphorlmager (Molecular Dynamics, Sunnyvale, Calif.).

Oligonucleotide activity is assayed by quantitation of STAT3 mRNA levels by real-time PCR (RT-PCR) using the ABI PRISM.TM. 7700 Sequence Detection System (PE-Applied Biosystems, Foster City, Calif.) according to manufacture's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in RT-PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or FAM, PE-Applied Biosystems, Foster City, Calif.) is attached to the 5′ end of the probe and a quencher dye (e.g., TAMRA, PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM.TM. 7700 Sequence Detection System. In each assay, a series of parallel reactions containing serial dilutions of MRNA from untreated control samples generates a standard curve that is used to quantitate the percent inhibition after antisense oligonucleotide treatment of test samples.

RT-PCR reagents are obtained from PE-Applied Biosystems, Foster City, Calif.. RT-PCR reactions are carried out by adding 25 μl PCR cocktail (1×TAQMAN® buffer A, 5.5 mM MgCl₂, 300 μM each of dATP, dCTP and dGTP, 600 μM of dUTP, 100 nM each of forward primer, reverse primer, and probe, 20 U RNase inhibitor, 1.25 units AMPLITAQ GOLD®, and 12.5 U MuLV reverse transcriptase) to 96 well plates containing 25 μl poly(A) mRNA solution. The RT reaction is carried out by incubation for 30 minutes at 48° C. following a 10 minute incubation at 95° C. to activate the AMPLITAQ GOLD®, 40 cycles of a two-step PCR protocol are carried out: 95° C. for 15 seconds (denaturation) followed by 60° C. for 1.5 minutes (annealing/extension) STAT3 PCR primers and a probe can be designed using commercial software (e.g. Oligo 5.0). The efficacy of said modified oligonucleotides is compared to the conventional oligonucleotides and it will be observed that the introduction of at least 2 juxtaposed universal bases improves the efficiency of antisense inhibition of STAT3 in these cell lines.

In addition, the effect of the oligonucleotides is analyzed by identifying the effect on BCL1 proliferation because BCL1 cells contain constitutively active STAT3, which is thought to be responsible for their proliferation. Approximately, 10⁵ BCL1 cells are incubated in 96-well plates in 200 μL complete RPMI following electroporation. Cultures are pulsed with 1 μCi of [³ H]-thymidine for the last 8 hours of culture and cells are harvested and analyzed for thymidine incorporation as described in Francis, D. A. et al. (Int. Immunol., 1995, 7, 151-161) 48 hours after electroporation. The efficacy of the modified oligonucleotides is compared to the conventional oligonucleotides and it will be observed that the introduction of at least 2 juxtaposed universal bases improves the efficiency of antisense inhibition of STAT3 in these cell lines and thereby significantly reduces the proliferation of the BCL1 cells.

The oligonucleotides described above are then tested in a mouse model. The mouse model for Rheumatoid arthritis is used as follows: Collagen-induced arthritis (CIA) is used as a murine model for arthritis (Mussener, A., et al., Clin. Exp. Immunol., 1997, 107, 485-493). Female DBA/1LacJ mice (Jackson Laboratories, Bar Harbor, Me.) between the ages of 6 and 8 weeks are used to assess the activity of TNFα antisense oligonucleotides.

On day 0, the mice are immunized at the base of the tail with 100 μg of bovine type II collagen which is emulsified in Complete Freund's Adjuvant (CFA). On day 7, a second booster dose of collagen is administered by the same route. On day 14, the mice are injected subcutaneously with 100 μg of LPS. Oligonucleotide is administered intraperitoneally daily (10 mg/kg bolus) starting on day-3 and continuing for the duration of the study. Weights are recorded weekly. Mice are inspected daily for the onset of CIA. Paw widths are rear ankle widths of affected and unaffected joints are measured three times a week using a constant tension caliper. Limbs are clinically evaluated and graded on a scale from 0-4 (with 4 being the highest). The above natural and modifed oligonucleotides are compared to a saline control. The modified antisense STAT3 oligonucleotide will be identified as effectively inhibiting the symptoms of rheumatoid arthritis in the mouse model.

The equivalent oligonucleotides to the mouse oligonucleotides are identified in the human sequence and modified. The modified oligonucleotides are used to treat inflammation and, in this case, rheumatoid arthritis as follows: a patient with rheumatoid arthritis is diagnosed by means known to one of skill in the art, including but not limited to: by symptoms, by the presence of the rheumatoid factor, by sedimentation rate, and by X-ray. A therapeutically effective amount of the modified antisense olignonucleotides is administered daily until the symptoms are decreased or completely abate. For example, a bolus of 10 mg/kg is administered. At this time, the treatment may be stopped or reduced in frequency or dosage. Alternatively, the antisense oligonucleotide may be administered to a patient who is identified as prone to or at risk for developing rheumatoid arthritis before the onset. The next example describes an approach that can be used to treat and/or prevent diseases associated with the expression of HER2.

### EXAMPLE 9

HER-2 (also known as c-neu, ErbB-2 and HER-2/neu) encodes a transmembrane receptor (also known as p185) with tyrosine kinase activity and is a member of the epidermal growth factor (EGF) family, and is related to the epidermal growth factor receptor (EGFR or HER-1). Overexpression of HER-2 is involved in various forms of cancer. Aberrant HER-2 gene expression is present in a wide variety of cancers and are most common in breast, ovarian and gastric cancers. HER-2 is overexpressed in 25-30% of all human breast and ovarian cancers. Levels of HER-2 overexpression correlate well with clinical stage of breast cancer, prognosis and metastatic potential. Overexpression of HER-2 is associated with lower survival rates, increased relapse rates and increased metastatic potential.

Others have disclosed antisense techniques to inhibit HER-2 activity and thereby treat and/or prevent HER-2-associated disease (See e.g., U.S. Pat. No. 5,968,748, herein incorporated by reference in its entirety.

The approach above can be improved by implementing the technology described herein. Accordingly, oligonucleotide sequences complementary to HER-2 are selected based upon their efficacy at down-regulating HER-2. Modifications are made to said oligonucleotides by incorporating blocks of at least 2 juxtaposed universal bases. For example, oligonucleotides known to down-regulate HER-2 are chosen and modified as disclosed herein. These included the following natural and modified oligonucleotides:

| | |
|---|---|
| Unmodified: | |
| GGTCAGGCAGGCTGTCCGGC | (SEQ ID NO:55) |
| GTCCCCACCGCCACTCCTGG | (SEQ ID NO:56) |
| GCATGGCAGGTTCCCCTGGA | (SEQ ID NO:57) |
| GTCCCCACCGCCACTCCTGG | (SEQ ID NO:58) |
| GTCCCCACCGCCACTCCTGG | (SEQ ID NO:59) |
| GTCCCCACCGCCACTCCTGG | (SEQ ID NO:60) |
| Modified: | |
| GGTCBBBCAGGCTGTCCGGC | (SEQ ID NO:61) |
| GTBBBCACCGCCABBBBTGG | (SEQ ID NO:62) |
| GCATGGCABBBBBBCCTGGA | (SEQ ID NO:63) |
| GTCCCCABBBBBBBBBCTGG | (SEQ ID NO:64) |
| GTBBBCACCBBCACTCBBGG | (SEQ ID NO:65) |
| GTCBBBBBCGCCACTCCTGG | (SEQ ID NO:66) |

SKOV3 cells are grown until 65-75% confluent. The cells are washed once with serum-free OPTI-MEM® medium (Life Technologies, Inc., Grand Island, N.Y.) and serum-free OPTI-MEM® containing 15 μg/ml of LIPOFECTIN® reagent (a 1:1 liposome formulation of the cationic lipid DOTMA and DOPE, Life Technologies, Inc.) was added. At that time, 300 nM of oligonucleotide is added and swirled vigorously. After a 4 hour incubation at 37° C., the solution is removed and fresh maintenance medium containing 10% fetal bovine serum was added. The cells are again incubated overnight at 37° C., after which the cells are assayed for HER-2 MRNA expression.

Total mRNA is extracted from the SKOV3 cells by washing cells twice with PBS and adding RNAZOL B® (Tel-Test, Inc., Friendswood, Tex.). An incubation at 4° C. for 5-30 minutes is done and the cells are scraped into an Eppendorf tube. This solution is frozen at −80° C. for 20 minutes, thawed and chloroform (200 μl/ml) is added. The solution is centrifuged at 12,000×g for 15 minutes at 4° C. and the aqueous layer is transferred to a clean Eppendorf tube. An equal volume of isopropanol is added and incubated at room temperature for 15 minutes. Another centrifugation at 12,000×g for 15 minutes at 4° C. is done. The pellet is washed with 500 μl of 75% ethanol and centrifuged at 7500×g for 5 minutes at 4° C. As much of the supernatant as possible is removed and the pellet is resuspended in double distilled water. The mRNA is resolved on a 1.0% agarose gel containing 3.0% formaldehyde and transferred to a nylon membrane. The membrane is hybridized with an asymmetric PCR-generated human HER-2 probe radiolabeled with [α-³² P]-dCTP (Dupont NEN Research Products, Boston, Mass.). The HER-2 probe is generated with the pTRI-erbB2-Human transcription template (Ambion, Austin, Tex.) using the GeneAMP PCR Reagent Kit (Perkin Elmer, Foster City, Calif.) and a T7 primer. The membrane is exposed to autoradiography film at −80° C. and the mRNA bands quantitated using a densitometer (Molecular Dynamics). Blots are stripped of radioactivity by boiling and then reprobed with a ³² P-labeled control probe which hybridized to G3PDH (Clontech Laboratories, Inc., Palo Alto, Calif.). The modified antisense HER-2 oligonucleotide will be identified as effectively inhibiting the expression of HER-2 in the cell line.

The modified oligonucleotides are used to treat cancer as follows: a patient with breast cancer or at risk for breast cancer is identified by methods known to one of skill in the art, for example, by identification of a lump, a family history of disease, and other risk factors. Alternatively, the overexpression of HER-2 may be identified in a specific patient. A therapeutically effective amount of the modified antisense olignonucleotides is administered daily until the symptoms are decreased or completely abate. For example a bolus of 10 mg/kg is administered intravenously. Alternatively, the antisense oligonucleotides may be administered locally to a lymph node and/or a lump or surrounding area. When a reduction in size of the tumor is identified or alternatively, when a biopsy identifies no abnormal cells, the treatment may be stopped or reduced in frequency or dosage. Alternatively, the antisense oligonucleotide may be administered to a patient who is identified as prone to or at risk for developing breast cancer before the onset. In the next example, an approach to inhibit the expression FAK so as to inhibit the proliferation of various types of cancers is described.

### EXAMPLE 10

FAK a non-receptor protein-tyrosine kinase localized to cell substratum-extracellular matrix (ECM) contact sites that function as part of a cytoskeletal-associated network of signaling proteins. Overexpression of FAK is involved in cancer progression. In addition, high levels of FAK correlates with invasiveness and metastatic potential in cancers, including but not limited to: colon tumors, breast tumors, and oral cancers.

Others have disclosed antisense techniques to inhibit FAK activity and thereby treat and/or prevent FAK-associated disease (See e.g., U.S. Pat. No. 6,133,031, herein incorporated by reference in its entirety).

The approach above can be improved by implementing the technology described herein. Accordingly, oligonucleotide sequences complementary to FAK are selected based upon their efficacy at down-regulating FAK. Modifications are made to said oligonucleotides by incorporating blocks of at least 2 juxtaposed universal bases. For example, oligonucleotides known to down-regulate FAK are chosen and modified as disclosed herein. These included the following natural and modified oligonucleotides:

| | |
|---|---|
| Unmodified: | |
| GGTCAGGCAGGCTGTCCGGC | (SEQ ID NO:67) |
| GTCCCCACCGCCACTCCTGG | (SEQ ID NO:68) |
| GCATGGCAGGTTCCCCTGGA | (SEQ ID NO:69) |
| GTCCCCACCGCCACTCCTGG | (SEQ ID NO:70) |
| GTCCCCACCGCCACTCCTGG | (SEQ ID NO:71) |
| GTCCCCACCGCCACTCCTGG | (SEQ ID NO:72) |
| Modified: | |
| GGTCBBBCAGGCTGTCCGGC | (SEQ ID NO:73) |
| GTBBBCACCGCCABBBBTGG | (SEQ ID NO:74) |
| GCATGGCABBBBBBCCTGGA | (SEQ ID NO:75) |
| GTCCCCABBBBBBBBBCTGG | (SEQ ID NO:76) |
| GTBBBCACCBBCACTCBBGG | (SEQ ID NO:77) |
| GTCBBBBBCGCCACTCCTGG | (SEQ ID NO:78) |

SKOV3 cells are grown until 65-75% confluent. The cells are washed once with serum-free OPTI-MEM® medium (Life Technologies, Inc., Grand Island, N.Y.) and serum-free OPTI-MEM® containing 15 μg/ml of LIPOFECTIN® reagent (a 1:1 liposome formulation of the cationic lipid DOTMA and DOPE, Life Technologies, Inc.) was added. At that time, 300 nM of oligonucleotide is added and swirled vigorously. After a 4 hour incubation at 37° C., the solution is removed and fresh maintenance medium containing 10% fetal bovine serum was added. The cells are again incubated overnight at 37° C., after which the cells are assayed for HER-2 mRNA expression.

Total mRNA is extracted from the SKOV3 cells by washing cells twice with PBS and adding RNAZOL B® (Tel-Test, Inc., Friendswood, Tex.). An incubation at 4° C. for 5-30 minutes is done and the cells are scraped into an Eppendorf tube. This solution is frozen at −80° C. for 20 minutes, thawed and chloroform (200 μl/ml) is added. The solution is centrifuged at 12,000×g for 15 minutes at 4° C. and the aqueous layer is transferred to a clean Eppendorf tube. An equal volume of isopropanol is added and incubated at room temperature for 15 minutes. Another centrifugation at 12,000×g for 15 minutes at 4° C. is done. The pellet is washed with 500 μl of 75% ethanol and centrifuged at 7500×g for 5 minutes at 4° C. As much of the supernatant as possible is removed and the pellet is resuspended in double distilled water. The mRNA is resolved on a 1.0% agarose gel containing 3.0% formaldehyde and transferred to a nylon membrane. The membrane is hybridized with an asymmetric PCR-generated human HER-2 probe radiolabeled with [α-³² P]-dCTP (Dupont NEN Research Products, Boston, Mass.). The HER-2 probe is generated with the pTRI-erbB2-Human transcription template (Ambion, Austin, Tex.) using the GeneAMP PCR Reagent Kit (Perkin Elmer, Foster City, Calif.) and a T7 primer. The membrane is exposed to autoradiography film at −80° C. and the mRNA bands quantitated using a densitometer (Molecular Dynamics). Blots are stripped of radioactivity by boiling and then reprobed with a ³² P-labeled control probe which hybridized to G3PDH (Clontech Laboratories, Inc., Palo Alto, Calif.). The modified antisense FAK oligonucleotide will be identified as effectively inhibiting the expression of FAK in the cell line.

The modified oligonucleotides are used to treat cancer as follows: a patient with breast cancer or at risk for breast cancer is identified by methods known to one of skill in the art, for example, by identification of a lump, a family history of disease, and other risk factors. Alternatively, the overexpression of FAK may be identified in a specific patient. A therapeutically effective amount of the modified antisense olignonucleotides is administered daily until the symptoms are decreased or completely abate. For example a bolus of 10 mg/kg is administered intravenously. Alternatively, the antisense oligonucleotides may be administered locally to a lymph node and/or a lump or surrounding area. When a reduction in size of the tumor is identified or alternatively, when a biopsy identifies no abnormal cells, the treatment may be stopped or reduced in frequency or dosage. Alternatively, the antisense may be administered to a patient who is identified as prone to or at risk for developing breast cancer before the onset. The next example describes an approach that can be used to treat and/or prevent a disease associated with the overexpression of TNF-α.

### EXAMPLE 11

TNF-α encodes a natural cytokine involved in the regulation of immune function and is implicated in infectious and inflammatory diseases, including but not limited to, insulin-dependent diabetes mellitis, rheumatoid arthritis, Crohn's disease, hepatitis, pancreatitis and atopic dermatitis. Others have disclosed antisense techniques to inhibit TNF-α activity and thereby treat and/or prevent TNFα-associated disease (See e.g., U.S. Pat. No. 6,228,642, herein incorporated by reference in its entirety).

The approach above can be improved by implementing the technology described herein. Accordingly, oligonucleotide sequences complementary to TNFα are selected based upon their efficacy at down-regulating TNFα. Modifications are made to said oligonucleotides by incorporating blocks of juxtaposed universal bases. For example, oligonucleotides known to down-regulate TNFα are chosen and modified as disclosed herein. These included the following natural and modified oligonucleotides:

| | |
|---|---|
| Unmodified: | |
| AGAGCTCTGTCTTTTCTCAG | (SEQ ID NO:79) |
| TCTTTGAGATCCATGCCGTT | (SEQ ID NO:80) |
| CTCCTCCCAGGTATATGGGC | (SEQ ID NO:81) |
| GTGAATTCGGAAAGCCCATT | (SEQ ID NO:82) |
| Modified: | |
| AGAGCTCBBBBBTTTCTCAG | (SEQ ID NO:83) |
| TCTTTGAGATCCBBBBCGTT | (SEQ ID NO:84) |
| CTBBBBCCAGGTATATGGGC | (SEQ ID NO:85) |
| GTGAATTCGGAAABBCCATT | (SEQ ID NO:86) |

The oligonucleotides are compared in vitro as follows: P388D1, mouse macrophage cells (obtained from American Type Culture Collection, Manassas, Va.) are cultured in RPMI 1640 medium with 15% fetal bovine serum (FBS) (Life Technologies, Rockville, Md.). At assay time, cells are at approximately 90% confluency. The cells are incubated in the presence of OPTI-MEM® medium (Life Technologies, Rockville, Md.), and the oligonucleotide formulated in LIPOFECTIN® (Life Technologies), a 1:1 (w/w) liposome formulation of the cationic lipid N-[1 -(2,3-dioleyloxy)propyl]-n,n,n-trimethylammonium chloride (DOTMA), and dioleoyl phosphotidylethanolamine (DOPE) in membrane filtered water. For an initial screen, the oligonucleotide concentration is from 10 to 100 nM in 3 μg/ml LIPOFECTIN®. Treatment is for four hours. After treatment, the medium is removed and the cells are further incubated in RPMI medium with 15% FBS and induced with LPS. mRNA is analyzed 2 hours post-induction with PMA. Total mRNA is isolated using the TOTALLY RNA™ kit (Ambion, Austin, Tex.), separated on a 1% agarose gel, transferred to HYBOND™ N+ membrane (Amersham, Arlington Heights, Ill.), a positively charged nylon membrane, and probed. A TNFα probe consists of the 502 bp EcoRI-HindIII fragment from BBG 56 (R&D Systems, Minneapolis, Minn.), a plasmid containing mouse TNFα cDNA. A glyceraldehyde 3-phosphate dehydrogenase (G3PDH) probe consists of the 1.06 kb HindIII fragment from pHcGAP (American Type Culture Collection, Manassas, Va.), a plasmid containing human G3PDH cDNA. The fragments are purified from low-melting temperature agarose, as described in Maniatis, T., et al., Molecular Cloning: A Laboratory Manual, 1989 and labeled with REDIVUE.TM. ³² P-dCTP (Amersham Pharmacia Biotech, Piscataway, N.J.) and PRIME-A-GENE® labelling kit (Promega, Madison, Wis.). mRNA is quantitated by a Phospholmager (Molecular Dynamics, Sunnyvale, Calif.).

Secreted TNFα protein levels are measured using a mouse TNFα ELISA kit (R&D Systems, Minneapolis, Minn. or Genzyme, Cambridge, Mass.). LIPOFECTIN® is added at a ratio of 3 μg/ml per 100 nM of oligonucleotide. The control includes LIPOFECTIN® at a concentration of 6 μg/ml. The efficacy of said modified oligonucleotides is compared to the conventional oligonucleotides and it will be observed that the introduction of juxtaposed universal bases improves the efficiency of antisense inhibition of TNFα in these cell lines.

The oligonucleotides are then tested in a mouse model of disease. The mouse model for Rheumatoid arthritis is used as follows: Collagen-induced arthritis (CIA) is used as a murine model for arthritis (Mussener, A., et al., Clin. Exp. Immunol., 1997, 107, 485-493). Female DBA/1LacJ mice (Jackson Laboratories, Bar Harbor, Me.) between the ages of 6 and 8 weeks are used to assess the activity of TNFα antisense oligonucleotides.

On day 0, the mice are immunized at the base of the tail with 100 μg of bovine type II collagen which is emulsified in Complete Freund's Adjuvant (CFA). On day 7, a second booster dose of collagen is administered by the same route. On day 14, the mice are injected subcutaneously with 100 μg of LPS. Oligonucleotide is administered intraperitoneally daily (10 mg/kg bolus) starting on day-3 and continuing for the duration of the study. Weights are recorded weekly. Mice are inspected daily for the onset of CIA. Paw widths are rear ankle widths of affected and unaffected joints are measured three times a week using a constant tension caliper. Limbs are clinically evaluated and graded on a scale from 0-4 (with 4 being the highest). The above natural and modifed oligonucleotides are compared to a saline control. The modified antisense TNFα oligonucleotide will be identified as more effectively inhibiting the symptoms of rheumatoid arthritis in the mouse model than the natural oligonucleotides.

The equivalent oligonucleotides to the mouse oligonucleotides are identified in the human sequence and modified. The modified oligonucleotides are used to treat inflammation and in this case rheumatoid arthritis as follows: a patient with rheumatoid arthritis is diagnosed by means known to one of skill in the art, including but not limited to: by symptoms, by the presence of the rheumatoid factor, by sedimentation rate, and by X-ray. A therapeutically effective amount of the modified antisense olignonucleotides is administered daily until the symptoms are decreased or completely abate. For example a bolus of 10 mg/kg is administered. At this time, the treatment may be stopped or reduced in frequency or dosage. Alternatively, the antisense may be administered to a patient who is idenified as prone to or at risk for developing rheumatoid arthritis before the onset. The next example describes the use of antisense oligonucleotides comprising at least two juxtaposed universal bases to inhibit the expresion of SDI genes and thereby induce the proliferation of cells in a subject.

### EXAMPLE 12

Cell senescence inhibitors, which are inhibitors of DNA synthesis produced in senescent cells (SDI), are identified from the sequence provided in U.S. Pat. No. 5,840,845 (herein incorporated by reference in its entirety). The inhibitor identified in the aforementioned patent plays a crucial role in the expression of the senescent phenotype. Antisense inhibitors of this gene (SDI) may be used to treat a disease that is characterised by the inhibition of senescence, such as aging skin cells, wound healing, and the recovery after bums. For such embodiments, the antisense agents may be formulated with antibiotics, anti-fungal agents, or the like, for topical or systemic administration. Such antisense and other inhibitor molecules of the present invention may be used to stimulate the proliferation of spermatocytes, or the maturation of oocytes in humans or animals, as well. Thus, the agents of the present invention may also be used to increase the fertility of a recipient.

Others have disclosed antisense techniques to inhibit this activity and thereby treat and/or prevent senescence-associated disease (See e.g., U.S. Pat. No. 5,840,845, herein incorporated by reference in its entirety).

The approach above can be improved by implementing the technology described herein. Accordingly, oligonucleotide sequences complementary to the senescence inhibitor are selected based upon their efficacy at down-regulating SDI. Oligonucleotides may be chosen to be complementary to the 3′ end or 5′ end or the gene, for example. Modifications are made to said oligonucleotides by incorporating blocks of at least 2 juxtaposed universal bases.

The modified oligonucleotides are used to treat bum wounds as follows: a patient with a bum wound is identified. Alternatively, the overexpression of the senescence inhibitor may be identified in a specific patient. A therapeutically effective amount of the modified antisense olignonucleotides is administered daily until the wound is healed and the skin begins to grow back. For example a bolus of 10 mg/kg is administered intradermally at the site of the wound. Alternatively, the antisense oligonucleotides may be administered intravenously if the burn wound covers too much of the body. Alternatively the oligonucleotides may be administered topically. When the skin has grown back or the wound has healed, the treatment may be stopped or reduced in frequency or dosage.

Within this application, unless otherwise stated, the techniques utilized may be found in any of several well-known references including: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), Berger et al., Guide to Molecular Cloning Techniques, Methods in Enzymology, Vol. 152, Academic Press, Inc., (1987); Davis et al., Basic Methods in Molecular Biology, Elsevier Science Publishing Co., Inc. (1986); Ausubel et al., Short Protocols in Molecular Biology, 2nd ed., John Wiley & Sons, (1992), Grinsted et al., Plasmid Technology, Methods in Microbiology, Vol. 21, Academic Press, Inc., (1988); Symonds et al., Phage Mu, Cold Spring Harbor Laboratory Press (1987), Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2.sup.nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). The basic principles of eukaryotic gene structure and expression are generally known in the art. (See for example Hawkins, Gene Structure and Expression, Cambridge University Press, Cambridge, UK, 1985; Alberts et al., The Molecular Biology of the Cell, Garland Press, New York, 1983; Goeddel, Gene Expression Technology, Methods in Enzymology, Vol. 185, Academic Press, Inc., (1991); Lewin, Genes VI, Oxford Press, Oxford, UK, 1998). Each of the above-mentioned references are hereby incorporated by reference in their entirety.

Although the invention has been described with reference to embodiments and examples, it should be understood that various modifications can be made without departing from the spirit of the invention. All references cited herein are hereby expressly incorporated by reference.

What is claimed is:

1. An improved antisense oligonucleotide, which comprises a domain that recruits an RNase, and inhibits the function of a gene associated with a human disease, wherein the improvement comprises the incorporation of at least two juxtaposed universal bases in said oligonucleotide.

2. The improved antisense oligonucleotide of claim 1, wherein the disease is a cancer.

3. The improved antisense oligonucleotide of claim 2, wherein the disease is a cancer characterised by an overexpression of BCL2.

4. The improved antisense oligonucleotide of claim 1, wherein the disease is melanoma.

5. The improved antisense oligonucleotide of claim 1, wherein the disease is a cancer characterised by an overexpression of a gene selected from the group consisting of STAT3, HER-2, and FAK.

6. The improved antisense oligonucleotide of claim 1, wherein the disease is an inflammatory disease characterised by an expression of TNF-α.

7. The improved antisense oligonucleotide of claim 1, wherein said oligonucleotide comprises at least 3 juxtaposed universal bases.

8. The improved antisense oligonucleotide of claim 1, wherein said oligonucleotide comprises at least 4 juxtaposed universal bases.

9. The improved antisense oligonucleotide of claim 1, wherein said oligonucleotide comprises at least 5 juxtaposed universal bases.

10. A pharmaceutical comprising the improved antisense oligonucleotide of claim 1 in conjunction with a pharmaceutically acceptable carrier.

11. A method of inhibiting the function of a gene associated with a human disease comprising contacting a cell containing said gene with the improved antisense oligonucleotide comprising at least two juxtaposed universal bases, whereby the function of the gene in said cell is inhibited.

12. The method of claim 11, wherein said gene is BCL2.

13. The method of claim 11, wherein said gene is selected from the group consisting of STAT3, HER-2, FAK, and TNF-α.

14. The method of claim 11, wherein said oligonucleotide comprises at least 3 juxtaposed universal bases.

15. The method of claim 11, wherein said oligonucleotide comprises at least 4 juxtaposed universal bases.

16. The method of claim 11, wherein said oligonucleotide comprises at least 5 juxtaposed universal bases.

17. A method of inhibiting the function of a gene associated with a human disease comprising:
providing an antisense oligonucleotide comprising a domain that recriuts an RNase and at least 2 juxtaposed universal bases;
contacting a cell that expresses said gene with said antisense oligonucleotide whereby said contact inhibits the function of said gene.

18. The method of claim 17, wherein said oligonucleotide comprises at least 3 juxtaposed universal bases.

19. The method of claim 17, wherein said oligonucleotide comprises at least 4 juxtaposed universal bases.

20. The method of claim 17, wherein said oligonucleotide comprises at least 5 juxtaposed universal bases.
